SEQUENCE LISTING (14 sequences)

SEQ ID NO: 1 - DNA, 10 bp, Artificial Sequence
  Primer
tgacaatagc

SEQ ID NO: 2 - DNA, 47 bp, Artificial Sequence
  Probe
acaaaatacctgtattcctcgcctgtccagggatctgctcttacaga

SEQ ID NO: 3 - DNA, 53 bp, Artificial Sequence
  Probe
ctaatctgtaagagcagatccctggacaggcgaagaatacaggtattttgtcc

SEQ ID NO: 4 - DNA, 49 bp, Artificial Sequence
  Probe
ggacaaaatacctgtattcctcgcctgtccagggatctgctcttacaga

SEQ ID NO: 5 - DNA, 53 bp, Artificial Sequence; identical to SEQ ID NO: 3
  Probe

SEQ ID NO: 6 - DNA, 49 bp, Artificial Sequence; identical to SEQ ID NO: 4
  Probe

SEQ ID NO: 7 - DNA, 53 bp, Artificial Sequence; identical to SEQ ID NO: 3
  Probe

SEQ ID NO: 8 - DNA, 10 bp, Artificial Sequence
  Primer
tgacaatagt

SEQ ID NO: 9 - DNA, 47 bp, Artificial Sequence
  Probe
acaaaatacctgtattccttgcctgtccagggatctgctcttacaga

SEQ ID NO: 10 - DNA, 53 bp, Artificial Sequence; identical to SEQ ID NO: 3
  Probe

SEQ ID NO: 11 - DNA, 49 bp, Artificial Sequence
  Probe
ggacaaaatacctgtattccttgcctgtccagggatctgctcttacaga

SEQ ID NO: 12 - DNA, 53 bp, Artificial Sequence; identical to SEQ ID NO: 3
  Probe

SEQ ID NO: 13 - DNA, 49 bp, Artificial Sequence; identical to SEQ ID NO: 11
  Probe

SEQ ID NO: 14 - DNA, 53 bp, Artificial Sequence; identical to SEQ ID NO: 3
  Probe

(19) United States
(12) Patent Application Publication
(10) Pub. No.: US 2007/0092880 A1
(43) Pub. Date: Apr. 26, 2007

(54) Invasive cleavage reaction with electrochemical readout

(76) Inventors: Donald M. Crothers, Northford, CT (US); Peggy S. Eis, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/564,660

(22) PCT Filed: Jul. 14, 2004
(86) PCT No.: PCT/US04/22465

§ 371 Date: Dec. 7, 2006

Related U.S. Application Data
(60) Provisional application No. 60/488,177, filed on Jul. 16, 2003.
(60) Provisional application No. 60/532,102, filed on Dec. 23, 2003.

(51) Int. Cl. C12Q 1/68; C12P 19/34

(52) U.S. Cl. 435/6; 435/91.2

(57) ABSTRACT

A universal tag assay is disclosed wherein at least one invasive cleavage reaction (ICR) is used to generate tagged molecules having identifier tags corresponding to target nucleotide sequences, and further wherein hybridization of any tagged molecule with a complementary detection probe on a universal detector indicates the presence of the corresponding target in the sample being assayed. Preferred embodiments include the use of ICR to generate molecules suitable for use in the universal tag assay to detect variant nucleotide sequences including single nucleotide polymorphisms (SNPs), allelic variants, and splice variants. Hybridization of tagged molecules to detection probes is preferably detected by electrochemical readout, in particular the use of ruthenium amperometry to detect hybridization of identifier tags to detection probes immobilized on a universal detector, preferably a universal chip having gold or carbon electrodes.

### FIELD OF THE INVENTION

The present invention relates to a universal tag assay wherein at least one invasive cleavage reaction (ICR) is used to generate tagged molecules having identifier tags corresponding to targets, and hybridization of any tagged molecule with a complementary detection probe on a universal detector indicates the presence of the corresponding target in the sample being assayed. In particular, the present invention relates to using ICR methods, alone or in combination with other methods, to detect target nucleotide sequences and to amplify signals derived from target nucleotide sequences. Preferred embodiments include the use of ICR to generate molecules suitable for use in the universal tag assay to detect variant sequences including single nucleotide polymorphisms (SNPs), allelic variants, and splice variants. Hybridization of tagged molecules to detection probes is preferably detected by electrochemical readout, in particular the use of ruthenium amperometry to detect hybridization of identifier tags to detection probes immobilized on a universal detector, preferably a universal chip having gold or carbon electrodes.

### BACKGROUND OF THE INVENTION

The invasive cleavage reaction (ICR) provides methods for detection and quantitative analysis of DNA or RNA. Assays using ICR do not amplify the target of interest but rather, generate and amplify an unrelated signal only in the presence of the correct target sequence. By avoiding target amplification, in particular exponential amplification processes such as PCR, ICR avoids the possibility of contamination and the resulting false positive signal. Because of the degree of sensitivity and discrimination of invasive cleavage assays, these assays can be used to detect subattomole levels of target nucleic acids within complex mixtures, and variant sequences such as single nucleotide polymorphisms (SNPs) can be detected directly from genomic DNA without the need for prior amplification of the target sequence. (Hall et al., 2000, Proc Natl Acad Sci USA 97:8272-8277; Stevens et al., 2001, Nuc Acids Res 29:e77; de Arruda et al., 2002, Expert Rev Mol Diagn 2:487-496).

ICR generates invasive cleavage structures in a target-dependent manner, and structure-specific enzymes cleave these structures to release a signal that may be detected or may be further amplified before being detected. Each ICR requires at least two synthetic oligonucleotides, one called a “probe”, “downstream probe” or “signal probe” and one called an “upstream oligonucleotide” or “invader oligonucleotide”. The probe has a 3′ portion complementary to a target and a 5′ portion that is usually unrelated to the target sequence. The 5′ portion of the probe that does not hybridize to the target forms a “5′ flap”. The upstream oligonucleotide anneals to the target 5′ (upstream) of the 5′ portion of the probe annealed to the target, and the probe and upstream oligonucleotide overlap, creating a bifurcated overlapping structure that is considered to resemble a structure generated during strand displacement DNA synthesis. (Steven et al., 2001, Nuc Acids Res 29:e77) The probe and upstream nucleotide often overlap by one (1) nucleotide, although a longer overlap can also be used. The bifurcated structure or “cleavage structure” is cleaved to release the 5′ flap of the probe, and this released probe 5′ flap then functions as a signal that can be detected or used in a subsequent ICR. Many probes can be cleaved for each copy of the target without temperature cycling, enabling sensitive, linear signal amplification.

ICR utilizes enzymes that cleave nucleic acid molecules at specific sites based on structure rather than sequence, as they are specific for the bifurcated cleavage structures formed by probes and upstream oligonucleotides binding to target nucleotide sequences. However, these enzymes can also be considered to cleave in a structure- and target sequence-specific manner, in that the bifurcated structure recognized and cleaved by these enzymes is formed only in the presence of target nucleotide sequence. Structure-specific 5′ nucleases, whose primary cellular function is believed to be processing of Okazaki fragments, are used to cleave the probe in bifurcated complex at a position one nucleotide (1 nt) 3′ of the end of the upstream oligonucleotide, thus releasing the unpaired 5′ flap (Stevens et al., 2001, Nuc Acids Res 29:e77; Bambara et al., 1997. J Biol Chem 272:4647-4650). Thermostable 5′ endonucleases from the structure-specific archaebacterial flap endonuclease (FEN) family are extensively used in invasive cleavage assays.

In a commercial embodiment, nucleases utilized by Third Wave Technologies, Inc. for their “Invader®” ICR technology are called “Cleavase®” enzymes. Use of Invader® technology and Cleavase® enzymes are extensively disclosed in the following U.S. Pat. Nos. 6,458,535; 6,372,424; 6,358,691; 6,355,437; 6,348,314; 6,214,545; 6,210,880; 6,194,149; 6,090,606; 6,090,543; 6,001,567; 5,994,069; 5,985,557; 5,888,780; 5,846,717; 5,843,669; 5,843,654; 5,837,450; 5,795,763; 5,719,028; 5,614,402; 5,541,311, the entire contents of each of which are hereby incorporated by reference.

The specificity of ICR derives from the requirement of coordinated action of the upstream oligonucleotide and probe, as the upstream oligonucleotide and probe must both bind to the target and overlap to form the bifurcated cleavage structure. If there is no overlap, then no cleavage structure is formed and the structure-specific 5′ endonuclease cannot cleave the bifurcated structure to release the 5′ flap. This fact has been exploited in assays to detect genetic mutations and polymorphisms, as even a single nucleotide mismatch positioned at the site of overlap will disrupt the overlap and block cleavage, allowing sensitive discrimination of SNPs and mutations. Lyamichev et al. disclose identification of SNPs and deletions by carrying out a single invasive cleavage assay. (Lyamichev et al., 1999, Nat Biotech 17:292-296).The sensitivity results from probe turnover, wherein multiple probes are cleaved per target molecule (Lyamichev et al., 1999, Nat Biotech 17:292-296; Lyamichev et al., 2000, Biochemistry 39:9523-9532; Reynaldo et al., 2000, J Mol Biol 297:511-520).

Serial ICR, also known as serial invasive signal amplification reaction (SISAR), combines at least two ICR assays in series to generate and amplify a detectable signal in the presence of target nucleotide sequence. (Hall et al., 2000, Proc Natl Acad Sci USA 97:8272-8277 and U.S. Pat. No. 5,994,069, the entire contents of each of which are hereby incorporated by reference). In serial ICR, the probe 5′ flap released from the probe in the first ICR is not detected directly. Rather, the 5′ flap released in the first ICR is used as an upstream oligonucleotide (an “invader” oligonucleotide) in a second ICR. The cleavage product of the second ICR then serves as a signal. Hall et al. describe serial ICR using fluorescence resonance energy transfer (FRET) to detect the second cleavage product, where the second ICR probe is labelled with two dyes, a donor fluorescent dye and a quenching acceptor dye. (Hall et al., 2000, Proc Natl Acad Sci USA 97:8272-8277). When the probe 5′ flap is released in the second ICR, the two dyes are separated, quenching is eliminated, and the enhanced fluorescence signal from the donor dye on the released probe 5′ flap is detected with appropriate instrumentation. SISAR combines high degrees of sensitivity and specificity to detect small numbers of targets. In SISAR, as described by Hall et al. (2000, Proc Natl Acad Sci USA 97:8272-8277), more than 10⁷ reporter molecules (released probe 5′ flaps) were generated for each molecule of target DNA in a 4-hour reaction period, such that the reaction can detect as few as 1000 targets with no prior target amplification. Hall et al. also demonstrate that SISAR can detect as few as 600 copies of a gene in samples of human genomic DNA and can discriminate single nucleotide polymorphisms (SNPs) of the gene using as little as 20 ng of human genomic DNA.

### SUMMARY OF THE INVENTION

The present disclosure provides methods and compositions for using at least one invasive cleavage reaction (ICR) for detecting target nucleotide sequences in a sample using a universal tag assay. The universal tag assay disclosed and claimed herein provides tags and probes and universal detectors for use in a universal tag assay that advantageously minimizes spurious signals without the need to employ special conditions or special reagents. Targets are detected using the universal tag assay of the present invention by generating tagged molecules having identifier tags corresponding to targets, incubating tagged molecules with a universal detector having detection probes, and measuring hybridization of identifier tags to complementary detection probes, where hybridization of an identifier tag to its complementary detection probe indicates the presence of the target corresponding to that identifier tag.

Additional aspects of the present invention are described in the following numbered paragraphs:

1. A method of detecting a target nucleotide sequence in a sample, comprising:
a) providing
   i) template comprising at least one target nucleotide sequence;
   ii) at least one probe oligonucleotide comprising a 3′ portion complementary to a portion of said template comprising target nucleotide sequence and a 5′ portion not complementary to said template comprising target nucleotide sequence;
   iii) at least one upstream oligonucleotide complementary to a portion of said template comprising target nucleotide sequence wherein said portion is 5′ to and partially overlapping the 3′ portion of said template comprising target nucleotide sequence complementary to said probe oligonucleotide; and
   iv) a cleaving agent;
b) mixing, in any order, said template comprising target nucleotide sequence, said probe oligonucleotide, said upstream oligonucleotide, and said cleavage means under reaction conditions such that said 3′ portion of said probe oligonucleotide is annealed to said template and said upstream oligonucleotide is annealed to said template so as to create a cleavage structure wherein said probe oligonucleotide and said upstream oligonucleotide overlap by at least one nucleotide;
c) cleaving said cleavage structure to release a 5′ flap, comprising cleaving said probe oligonucleotide at a position one nucleotide 3′ of the portion of said probe oligonucleotide that overlaps said upstream oligonucleotide, releasing said 5′ flap comprising said 5′ portion of said probe oligonucleotide not complementary to contiguous target nucleotide sequence and further comprising any overlapping nucleotide;
d) utilizing said 5′ flap as a reagent in at least one subsequent reaction to generate at least one tagged molecule comprising an identifier tag chosen to serve as an identifier for said target nucleotide sequence;
e) contacting said at least one tagged molecule with a universal detector comprising at least one complementary detection probe coupled to a detection means, wherein said complementary detection probe comprises sequence complementary to said identifier tag; and
f) measuring hybridization of said identifier tag to said complementary detection probe, wherein said hybridization indicates the presence of the corresponding target nucleotide sequence in said sample.

2. The method of Paragraph 1, wherein said template is DNA or RNA.

3. The method of Paragraph 2, wherein said template comprises any one of genomic DNA, polymerase chain reaction (PCR) product, cDNA, rolling circle (RC) amplification product, mRNA, or viral RNA.

4. The method of Paragraph 1, wherein said reaction conditions comprise a reaction temperature between approximately 40 and approximately 75 degrees Centigrade.

5. The method of Paragraph 1, wherein multiple probe oligonucleotides are cleaved and multiple 5′ flaps are released.

6. The method of Paragraph 1, wherein said method is used to detect variant sequences of said target nucleotide sequence.

7. The method of Paragraph 1, wherein said probe oligonucleotide and said upstream oligonucleotide overlap by one nucleotide.

8. The method of Paragraph 7, wherein said method is used to determine one or more polymorphic nucleotides in a single nucleotide polymorphism (SNP), said method comprising providing at least one probe oligonucleotide and at least one upstream oligonucleotide complementary to and overlapping at the polymorphic nucleotide of a first allele of said SNP, and further providing at least one probe oligonucleotide and at least one upstream oligonucleotide complementary to and overlapping at the polymorphic nucleotide of a second allele of said SNP, wherein said oligonucleotide probes and upstream oligonucleotides anneal to said template so as to create a distinct cleavage structure for each allele of said SNP, with the result that an allele-specific 5′ flap is released from each cleavage structure if the corresponding allele of said SNP is present in said sample and with the further result that each said allele-specific 5′ flap released from said cleavage structure generates an allele-specific tagged molecule, wherein each said allele-specific tagged molecule comprises the identifier tag chosen to serve as the identifier for the corresponding allele.

9. The method of Paragraph 8, wherein said SNP comprises more than two alleles, further comprising providing oligonucleotide probe and upstream oligonucleotides complementary to and overlapping at each said allele of said SNP.

10. The method of Paragraph 1, wherein a plurality of target nucleotide sequences in a sample are detected, said method comprising:
a) providing template comprising a plurality of target nucleotide sequences, and further providing at least one probe oligonucleotide and at least one upstream oligonucleotide complementary to a portion of template comprising each target nucleotide sequence of said plurality of target nucleotide sequences;
b) mixing said oligonucleotide probes and upstream oligonucleotides with said template under reaction conditions such that each oligonucleotide probe and upstream oligonucleotide will anneal to said template to create a distinct cleavage structure for each target nucleotide sequence, with the result that at least one distinct 5′ flap corresponding to each said target nucleotide sequence of said plurality of target nucleotide sequences is released from each distinct cleavage structure if the corresponding target nucleotide sequence is present in said sample;
c) utilizing each said distinct 5′ flap as a reagent in at least one subsequence reaction to generate at least one distinct tagged molecule corresponding to each said target nucleotide sequence of said plurality of target nucleotide sequences, wherein each said distinct tagged molecule comprises the identifier tag chosen to serve as an identifier to each corresponding target nucleotide sequence; and
d) measuring hybridization of each said identifier tag corresponding to each said target nucleotide sequence of said plurality of target nucleotide sequences, wherein each said hybridization indicates the presence of said corresponding target nucleotide sequence in said sample.

11. The method of Paragraph 1, wherein said cleaving agent is a 5′ endonuclease.

12. A method of detecting a target nucleotide sequence in a sample, comprising:
a) performing a first ICR as in steps a) to c) of Paragraph 1, releasing a first 5′ flap;
b) performing a second ICR comprising:
   i) providing a second template, a second probe oligonucleotide, and said first 5′ flap, wherein said second probe oligonucleotide comprises a 3′ portion complementary to a portion of said second template and a 5′ portion not complementary to said second template, and further wherein said first 5′ flap is 5′ to and partially overlapping the 3′ portion of said second template comprising target nucleotide sequence complementary to said second probe oligonucleotide;
   ii) mixing, in any order, said second template, said second probe oligonucleotide, and said first 5′ flap, under reaction conditions such that said 3′ portion of said second probe oligonucleotide is annealed to said second template and said first 5′ flap is annealed to said second template so as to create a cleavage structure wherein said second probe oligonucleotide and said first 5′ flap overlap by at least one nucleotide;
   iii) cleaving said cleavage structure to release a second 5′ flap, comprising cleaving said second probe oligonucleotide at a position one nucleotide 3′ of the portion of said second probe oligonucleotide that overlaps said first 5′ flap, releasing said second 5′ flap comprising said 5′ portion of said second probe oligonucleotide not complementary to contiguous target nucleotide sequence and further comprising any overlapping nucleotide;
c) utilizing said second 5′ flap as a reagent in at least one subsequent reaction to generate at least one tagged molecule comprising an identifier tag chosen to serve as an identifier for said target nucleotide sequence;
d) contacting said at least one tagged molecule with a universal detector comprising at least one complementary detection probe coupled to a detection means, wherein said complementary detection probe comprises sequence complementary to said identifier tag; and
e) measuring hybridization of said identifier tag to said complementary detection probe, wherein said hybridization indicates the presence of the corresponding target nucleotide sequence in said sample.

13. The method of Paragraph 12, wherein no additional cleavage means is provided for said second ICR.

14. The method of Paragraph 12, wherein an additional cleavage means is provided for said second ICR.

15. The method of Paragraph 12, wherein said second template and said second probe oligonucleotide are provided as a hairpin cassette.

16. The method of Paragraph 15, wherein said hairpin cassette is addressably labelled, such that said labelled hairpin cassette can be manipulated through a label-binding moiety.

17. The method of Paragraph 16, wherein said hairpin cassette is biotinylated.

18. The method of Paragraph 17, wherein said biotinylated hairpin cassette is contacted with streptavidin coupled to a solid support, such that said biotinylated hairpin cassette can be removed from the reaction mixture.

19. The method of Paragraph 12, wherein said tagged molecule comprises an identifier tag chosen to serve as an identifier for said target nucleotide sequence, and a development reagent.

20. The method of Paragraph 19, wherein said contacting said tagged molecule with said universal detector is carried out in the presence of a development reagent binding moiety to generate a functional development reagent, further wherein said measuring hybridization of said identifier tag to said complementary detection probe includes measuring the contribution of said functional development reagent to the signal being measured.

21. The method of Paragraph 20, wherein said development reagent binding moiety comprises an oligonucleotide complementary to said development reagent.

22. The method of Paragraph 21, wherein said development reagent binding moiety is attached to at least one said detection probe.

23. The method of Paragraph 12, wherein said utilizing said second 5′ flap as a reagent in at least one subsequent reaction to generate at least one tagged molecule comprises using said second 5′ flap is used as a polymerization primer for rolling circle (RC) amplification of at least one circular oligonucleotide, said circular oligonucleotide comprising sequence complementary to said second 5′ flap and sequence complementary to an identifier tag chosen to serve as an identifier for said target nucleotide sequence, such that said RC amplification generates at least one tagged molecule comprising multiple copies of said identifier tag.

24. The method of Paragraph 23, wherein said method is used to determine one or more polymorphic nucleotides in a single nucleotide polymorphism (SNP).

25. The method of Paragraph 23, wherein said method is used to detect a plurality of target nucleotide sequences in said sample.

26. The method of Paragraph 23, wherein said tagged molecule is trimmed to generate shorter tagged molecules comprising one copy of said identifier tag.

27. The method of Paragraph 12, further comprising transcribing said second 5′ flap to generate RNA tagged molecules, wherein said method comprises:
a) providing at least one distinct second 5′ flap comprising sequence complementary to the identifier tag chosen to serve as the identifier for each said target nucleotide sequence in said sample;
b) contacting each said distinct second 5′ flap with a template comprising a double stranded RNA polymerase promoter and a single stranded portion of sequence complementary to said second 5′ flap;
c) allowing each said second 5′ flap to anneal to said sequence complementary to said second 5′ flap;
d) ligating each said annealed second 5′ flap to contiguous sequence;
e) transcribing each said annealed second 5′ flap in the presence of RNA polymerase, generating at least one RNA tagged molecule comprising the identifier tag for each said target nucleotide sequence in said sample; and
f) contacting said at least one tagged RNA molecule with a universal detector comprising at least one complementary detection probe coupled to a detection means, wherein each said complementary detection probe comprises sequence complementary to each said identifier tag, and measuring hybridization of each said identifier tag to each said complementary detection probe, wherein said hybridization indicates the presence of the corresponding target nucleotide sequence in the sample.

28. The method of Paragraph 27, wherein said template comprising a double stranded RNA polymerase promoter and a single stranded portion of sequence complementary to said second 5′ flap is a hairpin cassette.

29. The method of Paragraph 28, wherein said hairpin cassette is addressably labelled, such that said labelled hairpin cassette can be manipulated through a label-binding moiety.

30. The method of Paragraph 29, wherein said hairpin cassette is biotinylated.

31. The method of Paragraph 30, wherein said biotinylated hairpin cassette is contacted with streptavidin coupled to a solid support, such that said biotinylated hairpin cassette can be removed from the reaction mixture.

32. The method of Paragraph 27, wherein said method is used to determine one or more polymorphic nucleotides in a single nucleotide polymorphism (SNP).

33. The method of Paragraph 27, wherein said method is used to detect a plurality of target nucleotide sequences in said sample.

34. The method of Paragraph 12, further comprising transcribing said second 5′ flap to generate RNA tagged molecules, wherein said method comprises:
a) providing at least one distinct second 5′ flap corresponding to each said target nucleotide sequence in said sample;
b) contacting each said distinct second 5′ flap with at least one single stranded template, wherein each template comprises a portion of sequence complementary to one said distinct second 5′ flap, a portion of sequence encoding one strand of RNA polymerase promoter, and at least one copy of the identifier tag chosen to serve as the identifier for said target nucleotide sequence corresponding to said distinct second 5′ flap;
c) allowing each said second 5′ flap to anneal to said template sequence complementary to said second 5′ flap, forming a double-stranded DNA polymerase binding site;
d) generating a double stranded copy of said template by DNA polymerase binding to said double stranded site and extension of said annealed second 5′ flap;
e) contacting said double stranded copy of said template with RNA polymerase, allowing RNA polymerase to transcribe sequence downstream of said RNA polymerase promoter, thereby generating RNA tagged molecules comprising the identifier tag for each said target nucleotide sequence in said sample; and
f) contacting said at least one tagged RNA molecule with a universal detector comprising at least one complementary detection probe coupled to a detection means, wherein each said complementary detection probe comprises sequence complementary to each said identifier tag, and measuring hybridization of each said identifier tag to each said complementary detection probe, wherein said hybridization indicates the presence of the corresponding target nucleotide sequence in the sample.

35. The method of Paragraph 34, wherein said method is used to determine one or more polymorphic nucleotides in a single nucleotide polymorphism (SNP).

36. The method of Paragraph 34, wherein said method is used to detect a plurality of target nucleotide sequences in said sample.

37. A method of detecting a target nucleotide sequence in a sample comprising:
performing an invasive cleavage reaction wherein said invasive cleavage reaction releases a first 5′ flap only if said target nucleotide sequence is present in said sample;
generating a nucleic acid tag if said invasive cleavage reaction has released said first 5′ flap; and
detecting the presence of said nucleic acid tag by detecting the hybridization of said nucleic acid tag to a nucleic acid probe which is complementary to said nucleic acid tag.

38. The method of Paragraph 37, wherein the step of generating said nucleic acid tag comprises:
performing a second invasive cleavage reaction, wherein said second invasive cleavage reaction releases a second 5′ flap only if said first 5′ flap is present; and
performing a rolling circle amplification reaction using said second 5′ flap as a primer, wherein the product of said rolling circle amplification comprises said nucleic acid tag.

39. The method of Paragraph 37, wherein the step of generating said nucleic acid tag comprises
performing a second invasive cleavage reaction, wherein said second invasive cleavage reaction releases a second 5′ flap only if said first 5′ flap is present; and
performing a transcription reaction, said transcription reaction comprising extending said 5′ flap to generate a doublestranded nucleic acid comprising a promoter and initiating transcription from said promoter, wherein the resulting transcription product comprises said nucleic acid tag.

40. The method of Paragraph 37, wherein hybridization of said nucleic acid tag to said nucleic acid probe is detected by fixing said nucleic acid probe to a support, contacting said support with said nucleic acid tag, and measuring an electrical signal on said support which is indicative of hybridization of said nucleic acid tag to said nucleic acid probe.

41. The method of Paragraph 37, wherein said detecting step comprises ligating said first 5′ flap to a probe comprising a nucleotide sequence which forms a hairpin structure and detecting the hybridization of said nucleic acid tag to said probe.

42. The method of Paragraph 37, wherein said step of generating said nucleic acid tag comprises:
hybridizing a rolling circle probe/flap template to a rolling circle probe, wherein said rolling circle probe comprises a nucleotide sequence complementary to said nucleic acid tag and wherein said rolling circle probe/flap template is a nucleic acid comprising a nucleotide sequence complementary to said released first 5′ flap and nucleotide sequences complementary to nucleotide sequences in the terminal regions of said rolling circle probe;
ligating said released first 5′ flap to said rolling circle probe, thereby generating a circular nucleic acid; and
extending said rolling circle probe/flap template with a polymerase.

43. The method of Paragraph 37, wherein said step of generating said nucleic acid tag comprises:
hybridizing a rolling circle probe/flap template to a rolling circle probe, wherein said rolling circle probe comprises a nucleotide sequence complementary to said nucleic acid tag and wherein said rolling circle probe/flap template is a nucleic acid comprising a nucleotide sequence complementary to said released first 5′ flap, nucleotide sequences complementary to nucleotide sequences in the terminal regions of said rolling circle probe, and a promoter from which an RNA polymerase can initiate transcription;
ligating said released first 5′ flap to said rolling circle probe, thereby generating a circular nucleic acid; and
allowing transcription to initiate at said promoter such that a transcription product comprising said nucleic acid tag is generated.

44. The method of Paragraph 37, wherein said step of generating said nucleic acid tag comprises:
hybridizing a rolling circle probe/flap template to a rolling circle probe, wherein said rolling circle probe comprises a nucleotide sequence complementary to said nucleic acid tag and a nucleotide sequence complementary to a promoter from which an RNA polymerase can initiate transcription and wherein said rolling circle probe/flap template is a nucleic acid comprising a nucleotide sequence complementary to said released first 5′ flap and nucleotide sequences complementary to nucleotide sequences in the terminal regions of said rolling circle probe;
ligating said released first 5′ flap to said rolling circle probe, thereby generating a circular nucleic acid;
extending said rolling circle probe/ flap template across said nucleotide sequence complementary to said promoter; and
allowing transcription to initiate at said promoter such that a transcription product comprising said nucleic acid tag is generated.

45. The method of Paragraph 42, wherein said rolling circle probe/flap template is linked to the surface of a universal chip, such that said step of extending said hybridized nucleic acid comprises extending the 3′ terminus of said rolling circle probe/flap template.

46. A method of detecting a target nucleotide sequence in a sample comprising:
obtaining a nucleic acid probe comprising a 5′ region and a 3′ region wherein said 5′ region comprises a first sequence which is complementary to a first portion of said target nucleotide sequence and a second sequence 5′ of said first sequence which is not complementary to said target nucleotide sequence and wherein said 3′ region comprises a third sequence which is complementary to a second portion of said target nucleotide sequence;
contacting said sample with said nucleic acid probe under conditions in which said first sequence in said 5′ region hybridizes to said first portion of said target nucleotide sequence, said second sequence in said 5′ region of said nucleic acid probe forms a 5′ flap, and said third sequence in said 3′ region hybridizes to said second portion of said target nucleotide sequence;
cleaving said 5′ flap from said nucleic acid probe;
ligating the 5′ end of said cleaved nucleic acid probe to the 3′ end of said cleaved nucleic acid probe to generate a circular ligation product; and
detecting said circular ligation product.

47. The method of Paragraph 46, wherein said 5′ region of said nucleic acid probe lacks a phosphate on its 5′ end.

48. The method of Paragraph 46, wherein said first portion of said target nucleotide sequence is immediately adjacent to said second portion of said target nucleotide sequence.

49. The method of Paragraph 46, wherein said nucleic acid probe further comprises a nucleotide sequence complementary to an identifier tag.

50. The method of Paragraph 46 wherein said target nucleotide sequence comprises a polymorphic nucleotide and said nucleic acid probe comprises a nucleotide complementary to one allele of said polymorphic nucleotide.

51. The method of Paragraph 50, wherein said nucleotide complementary to said one allele of said polymorphic nucleotide is present in said first sequence of said 5′ region.

52. The method of Paragraph 50, wherein said nucleotide complementary to said one allele of said polymorphic nucleotide is present in said third sequence in said 3′ region.

53. The method of Paragraph 52, wherein said nucleotide complementary to said one allele of said polymorphic nucleotide is present at the 3′ terminus of said 3′ region.

54. The method of Paragraph 50, wherein said nucleotide complementary to said one allele of said polymorphic nucleotide is within about 5 nucleotides of the ligation site.

55. The method of Paragraph 46, wherein said target nucleotide sequence comprises a mutation which gives rise to cancer.

56. The method of Paragraph 46, wherein said target nucleotide sequence comprises a mutation which is present at a level of about one part in 100 or less.

57. The method of Paragraph 46, wherein said target nucleotide sequence comprises a mutation which is present at a level of about one part in 10,000 or less.

58. The method of Paragraph 46 wherein said detecting step comprises hybridizing a primer to said circular ligation product and performing a rolling circle amplification procedure to generate a rolling circle amplification product.

59. The method of Paragraph 46, wherein said detecting step comprises detecting the presence of the cleaved 5′ flap.

60. The method of Paragraph 46, wherein said sample is a DNA or RNA sample.

61. The method of Paragraph 46, wherein said sample comprises any one of genomic DNA, polymerase chain reaction (PCR) product, cDNA, rolling circle (RC) amplification product, mRNA, or viral RNA.

62. The method of Paragraph 46, wherein said target nucleotide sequence comprises a SNP.

63. The method of Paragraph 46, wherein said method is used to detect a plurality of target nucleotide sequences in said sample.

64. The method of Paragraph 58, wherein said rolling circle amplification product is trimmed.

65. The method of Paragraph 58, wherein said nucleic acid probe comprises a sequence complementary to an identifier tag such that said rolling circle amplification product contains a plurality of copies of said identifier tag.

66. The method of Paragraph 65, wherein said rolling circle amplification product is trimmed to generate shorter tagged molecules comprising one copy of said identifier tag.

67. A method of detecting a target nucleotide sequence in a sample comprising:
obtaining a nucleic acid probe comprising a 5∝ region and a 3′ region wherein said 5′ region comprises a first sequence which is complementary to a first portion of said target nucleotide sequence and a second sequence 5′ of said first sequence which is not complementary to said target nucleotide sequence and wherein said 3′ region comprises a third sequence which is complementary to a second portion of said target nucleotide sequence;
contacting said sample with said nucleic acid probe under conditions in which said first sequence in said 5′ region hybridizes to said first portion of said target nucleotide sequence and said second sequence in said 5′ region of said nucleic acid probe forms a 5′ flap;
cleaving said 5′ flap from said nucleic acid probe; and
detecting said cleaved flap.

68. The method of Paragraph 67, wherein the step of detecting said cleaved flap comprises using said cleaved flap as a primer in a rolling circle amplification procedure.

69. The method of Paragraph 67, wherein said cleaved flap comprises a promoter or a sequence complementary to a promoter and the step of detecting said cleaved flap comprises initiating transcription from said promoter.

70. A method of detecting a target nucleotide sequence in a sample comprising:
obtaining a first nucleic acid probe comprising a first sequence which is complementary to a first portion of said target nucleotide sequence and a second sequence 5′ of said first sequence which is not complementary to said target nucleotide sequence;
obtaining a second nucleic acid probe comprising a third sequence which is complementary to a second portion of said target nucleotide sequence;
contacting said sample with said first nucleic acid probe and said second nucleic acid probe under conditions in which said first sequence in said first nucleic acid probe hybridizes to said first portion of said target nucleotide sequence, said second sequence in said first nucleic acid probe forms a 5′ flap, and said third sequence in said second nucleic acid probe hybridizes to said second portion of said target nucleotide sequence;
cleaving said 5′ flap from said first nucleic acid probe;
ligating the 5′ end of said cleaved first nucleic acid probe to the 3′ end of said second nucleic acid probe to generate a ligation product; and
detecting said ligation product.

71. The method of Paragraph 70, wherein said first nucleic acid probe lacks a phosphate on its 5′ end.

72. The method of Paragraph 70, wherein said first portion of said target nucleotide sequence is immediately adjacent to said second portion of said target nucleotide sequence.

73. The method of Paragraph 70, wherein said first nucleic acid probe one strand of a promoter.

74. The method of Paragraph 73, wherein said detecting step comprises initiating transcription from said promoter and detecting a transcription product resulting from said transcription.

75. The method of Paragraph 73, wherein said second nucleic acid probe further comprises a nucleotide sequence complementary to an identifier tag.

76. The method of Paragraph 70 wherein said target nucleotide sequence comprises a polymorphic nucleotide and said nucleic acid probe comprises a nucleotide complementary to one allele of said polymorphic nucleotide.

77. The method of Paragraph 76, wherein said nucleotide complementary to said one allele of said polymorphic nucleotide is present in said first sequence of said first nucleic acid probe.

78. The method of Paragraph 76, wherein said nucleotide complementary to said one allele of said polymorphic nucleotide is present in said third sequence in said second nucleic acid probe.

79. The method of Paragraph 78, wherein said nucleotide complementary to said one allele of said polymorphic nucleotide is present at the 3′ terminus of said second nucleic acid probe.

80. The method of Paragraph 76, wherein said nucleotide complementary to said one allele of said polymorphic nucleotide is within about 5 nucleotides of the ligation site.

81. The method of Paragraph 70, wherein said target nucleotide sequence comprises a mutation which gives rise to cancer.

82. The method of Paragraph 70, wherein said target nucleotide sequence comprises a mutation which is present at a level of about one part in 100 or less.

83. The method of Paragraph 70, wherein said target nucleotide sequence comprises a mutation which is present at a level of about one part in 10,000 or less.

84. The method of Paragraph 70, wherein said detecting step comprises detecting the presence of the cleaved 5′ flap.

85. The method of Paragraph 70, wherein said sample is a DNA or RNA sample.

86. The method of Paragraph 70, wherein said sample comprises any one of genomic DNA, polymerase chain reaction (PCR) product, cDNA, rolling circle (RC) amplification product, mRNA, or viral RNA.

87. The method of Paragraph 70, wherein said target nucleotide sequence comprises a SNP.

88. The method of Paragraph 70, wherein said method is used to detect a plurality of target nucleotide sequences in said sample.

89. A method of detecting a target nucleotide sequence in a sample comprising:
obtaining a first nucleic acid probe comprising a first sequence which is complementary to a first portion of said target nucleotide sequence and a second sequence 5′ of said first sequence which is not complementary to said target nucleotide sequence;
obtaining a second nucleic acid probe comprising a third sequence which is complementary to a second portion of said target nucleotide sequence;
contacting said sample with said first nucleic acid probe and said second nucleic acid probe under conditions in which said first sequence in said first nucleic acid probe hybridizes to said first portion of said target nucleotide sequence, said second sequence in said first nucleic acid probe forms a 5′ flap, and said third sequence in said second nucleic acid probe hybridizes to said second portion of said target nucleotide sequence;
cleaving said 5′ flap from said nucleic acid probe; and
detecting said cleaved flap.

90. The method of Paragraph 89, wherein the step of detecting said cleaved flap comprises using said cleaved flap as a primer in a rolling circle amplification procedure.

91. The method of Paragraph 89, wherein said cleaved flap comprises a promoter or a sequence complementary to a promoter and the step of detecting said cleaved flap comprises initiating transcription from said promoter.

### BRIEF DESCRIPTION OF THE DRAWINGS

FIG. 1. Detection of single nucleotide polymorphisms using invasive cleavage reaction (ICR) and rolling circle (RC) amplification to produce tagged molecules having an identifier tag. As illustrated here, a target single nucleotide polymorphism (SNP) may have T or G at the polymorphic position. Step 1. First and second ICR. Probes specific for the T allele and the G allele and upstream oligonucleotides hybridize to template and cleavage with a 5′ nuclease releases the 5′ flaps of the probes. The cleaved 5′ flaps function as upstream oligonucleotides in a second ICR that uses biotinylated ICR hairpin cassettes. The second ICR occurs simultaneously with the first ICR and the same 5′ nuclease releases the hairpin flaps from the ICR hairpin cassettes. Step 2. Removal of hairpin cassettes. Both uncleaved and cleaved ICR hairpin cassettes can be removed via the 3′-biotin using streptavidin-coated (SA) beads or plates. This would prevent competition by the uncleaved ICR hairpin cassette 5′ flaps for the flap-binding sites present in the circular RC probes (step 3). Step 3. Signal amplification and generation of tagged molecules. The 5′ flaps released from hairpin cassettes function as polymerization primers for pre-circularized RC probes containing identifier tag complements. RC amplification generates multimers containing distinct identifier tags corresponding to each allele: P1 tag for the T allele; P2 tag for the G allele. Step 4. Detection. Tagged molecules generated by RC amplification are incubated with a universal detector in the presence of a ruthenium complex such as Ru(NH₃)⁶⁺, and hybridization is detected electrochemically. Signal from both C1 probe and C2 probe indicates the sample was heterozygous for the SNP, having both the T allele and the G allele present. Signal only from C1 indicates the sample was homozygous for the T allele. Signal only from C2 indicates the sample was homozygous for the G allele.

FIG. 2. Detection of single nucleotide polymorphisms using invasive cleavage reaction (ICR), ligation, and rolling circle (RC) amplification to produce tagged molecules having an identifier tag. Step 1. ICR. Similar to FIG. 1, an invasive cleavage reaction performed at the SNP site releases two different sequence 5′ flaps if the sample is from a heterozygote containing both alleles of the SNP. Probes (and released 5′ flaps) contain a 5′ phosphate and a 3′ hydroxyl. Step 2. Ligation. Cleaved 5′ flaps (1 and/or 2), which contain a 5′ phosphate and a 3′ hydroxyl, hybridize to their respective RC probe/flap templates and are specifically ligated thereto to form circularized RC probes, which can then be used as substrates in RC amplification reactions. Step 3. RC Amplification. RC amplification is performed on circularized RC probes to produce multimeric RNA or DNA containing distinct identifier tag sequences. Step 4. Detection. Performed as described in FIG. 1, Step 4.

FIG. 3. Detection of single nucleotide polymorphisms using integrated ICR probes, ligation, and RC amplification to produce tagged molecules having an identifier tag. An integrated ICR probe comprising both the upstream oligonucleotide and the downstream probe enables the use of two allele-specific enzymatic steps. Step 1. ICR. 5′ nuclease cleavage of the 5′ arm leaves a 5′ phosphate. Step 2. Ligation. Ligation of the cleavage product into a circle with a ligase. Step 3. RC Amplification. RC amplification of the SNP-specific created circles is performed to generate RC products. Step 4. Detection. Performed as described in FIG. 1, Step 4.

FIG. 4A. Detection of single nucleotide polymorphisms using integrated ICR probes

FIG. 4A shows one embodiment employing a circularizable integrated ICR probe comprising both the upstream oligonucleotide and the downstream probe is hybridized to the sample DNA. In the embodiment of FIG. 4A, the integrated ICR probe lacks a phosphate at its 5′ end. If the sample DNA contains the target nucleotide sequence, a portion of the 5′ region of the integrated ICR probe will hybridize to the target nucleotide sequence while the 5′ end of the integrated ICR probe forms a 5′ flap. The 3′ region of the integrated ICR probe will hybridize perfectly to the target nucleotide sequence if the target nucleotide sequence contains one allele at the polymorphic position but will contain a mismatch at the 3′ end if the target nucleotide sequence contains the other allele at the polymorphic position. Cleavage will occur efficiently if the 3′ region of the integrated ICR probe is perfectly complementary to the target nucleotide sequence but will be inefficient if there is a mismatch at the 3′ end. Ligation of the ends of the cleaved integrated ICR probe to generate a circular molecule will occur efficiently if the 3′ region of the integrated ICR probe is perfectly complementary to the target nucleotide sequence but will fail if there is a mismatch at the 3′ end. Rolling circle amplification is performed on the circularized molecules and the amplification products are detected.

FIG. 4B. FIG. 4B illustrates the efficient cleavage and ligation of the integrated ICR probe when the 3′ region is perfectly complementary to the target nucleotide sequence.

FIG. 4C. FIG. 4C illustrates the inefficient cleavage and ligation of the integrated ICR probe when the 3′ end is mismatched with the target nucleotide sequence.

FIG. 4D illustrates an embodiment in which the method is performed using two distinct oligonucleotides.

FIG. 5. RC amplification and ICR using development reagent. Step 1. RC amplification. Linear or exponential RC amplification is performed on genomic DNA. Steps 2A, 2B. ICR. Performed as described in FIG. 1, Step 1 except that the probe 5′ flaps contain additional oligonucleotide sequence or chemical moiety that enables binding to a development reagent. ICR is performed at the amplified SNP site (Step 2A) or at the junction between the generic sequence (distinct for a particular RC probe) and target-specific sequence (Step 2B). Step 3. Detection using development reagents. Tagged molecules generated by releasing 5′ flaps from hairpin cassettes contain an identifier tag and a sequence/chemical moiety that can bind development reagent to enhance electrochemical detection. Tagged molecules are incubated with a universal detector in the presence of a ruthenium complex such as Ru(NH₃)⁶⁺, and hybridization is detected electrochemically.

FIG. 6. Serial ICR using development reagent. Step 1. ICR. Performed as described in FIG. 1, Step 1 on genomic DNA. Steps 2. Removal of uncleaved ICR hairpin cassettes. Steptavidin (SA) coated beads or plate is used to removed uncleaved ICR hairpin cassettes via a 3′-biotin moiety. Step 3. Detection using development reagents. Detection is performed as described in FIG. 5, Step 3.

FIG. 7. Detection of target sequences with ICR and ligation on chip probes. Step 1. ICR. ICR is performed as described in FIG. 1, Step 1. If desired, the probe may contain a 3′ non-complementary sequence for use with a development reagent. Cleavage of the probe generates a target-specific region (TSR) that possesses a 5′-phophate. Step 2. Ligation on chip. The target-specific region of the cleaved probe hybridizes to its complementary sequence on the chip-bound probe. The chip probe is configured to possess a hairpin structure on its 3′ end, which, after hybridization of the cleaved 3′ end of the ICR probe, enables ligation of the chip probe to the cleaved 3′ TSR of the ICR probe. Step 3. Detection Detection is performed as described above.

FIG. 8. ICR followed by transcription. As illustrated here, a target SNP may have T or G at the polymorphic position. Step 1. First and second ICR. Performed as described in FIG. 1, Step 1 except ICR hairpin cassettes' 5′ flaps correspond to C1 tag and C2 tag sequences. Step 2. Ligation to transcription hairpin cassettes. The 5′ flaps released from the second ICR are ligated to biotinylated transcription hairpin cassettes containing T7 promoter sites. Step 3. Transcription. Transcription hairpin cassettes are captured onto a streptavidin surface and stringent washing is used to remove uncleaved, unligated ICR hairpin cassettes to reduce background. Addition of T7 polymerase generates single stranded RNA tagged molecules from cassettes having a ligated tag sequence. Step 4. Detection. Hybridization of RNA tagged molecules to complementary detection probes is detected by electrochemical readout as described in FIG. 1, Step 4.

FIG. 9. ICR followed by polymerization and transcription. As illustrated here, a target SNP may have T or G at the polymorphic position. Step 1. First ICR. Allele-specific upstream oligonucleotides and probes hybridize to template (genomic DNA or PCR product) and cleavage releases the 5′ flap of the probe. If a serial ICR is performed, the cleaved 5′ flaps from the first ICR function as upstream oligonucleotides in a second ICR using hairpin cassettes. ICR or serial ICR may be performed using any of the methods of FIGS. 1-8. Step 2. Polymerization and transcription. 5′ flaps released from the ICR or the second ICR of a serial ICR hybridize to flap/T7/tag templates and serve as polymerization primers. Polymerization generates double-stranded molecules containing a T7 promoter site, flap, and tag sequences. Step 3. Addition of T7 RNA polymerase generates single stranded RNA tagged molecules. Step 4. Detection. Hybridization of RNA tagged molecules to complementary detection probes is detected by electrochemical readout as described in FIG. 1, Step 4.

### DETAILED DESCRIPTION OF THE PREFERRED EMBODIMENT

The present disclosure provides methods and compositions for detecting target nucleotide sequences in a sample using invasive cleavage reaction (ICR) methods, alone or in combination with other methods, to generate tagged molecules suitable for use in a universal tag assay as disclosed in U.S. Provisional Application 60/424,656, the entire contents of which are hereby incorporated by reference. The present disclosure provides ICR methods, alone or in combination with other methods, for detecting target nucleotide sequences including variant sequences such as polymorphisms, allelic variants, insertions, deletions, and splice variants. Tagged molecules for use in a universal tag assay may be generated by one or more steps that include at least one target-dependent ICR in which a cleavage structure is formed on a template containing target nucleotide sequence and the cleavage product does not contain target nucleotide sequence or its complement. The cleavage product may be detected directly, or may be used in one or more subsequent reactions. The cleavage product of target-dependent ICR may be used as a reagent in subsequent reactions to generate at least one tagged molecule containing an identifier tag chosen to serve as the identifier for that target. Alternately, the cleavage product of target-dependent ICR may include an identifier tag. Further provided are methods for detecting the presence of a target nucleotide sequence using a universal tag assay. Preferably, electrochemical readout methods are used to measure results of the universal tag assay. More preferably, results of the universal tag assay are measured using ruthenium amperometry.

ICR can be performed on any DNA or RNA, including but not limited to genomic DNA, cDNA, PCR product, ligation product (LCR, OLA), mRNA, tRNA, rRNA, siRNA, and viral RNA. Use of ICR enables highly specific single-base discrimination. Advantageously, ICR using RNA targets permits detection of alternatively-spliced variants, and direct quantitation of specific RNAs as disclosed in Eis et al. 2001 (Nature Biotechnol 19:673-676).

After cleavage, the released 5′ flap contains generic sequences, e.g. molecules cleaved from the portion of a probe that is not complementary to a target, or tagged molecules containing identifier tags suitable for use with a universal detector in a universal tag assay. ICR reaction products also include the target-specific region of the probe (i.e. the portion which hybridizes to the target polynucleotide), which contains a 5′ phosphate. In addition, probes and upstream oligonucleotides can optionally contain generic or interchangeable sequence, depending on the assay. For example, the probes and upstream oligonucleotides used to determine the polymorphic nucleotide of an SNP may differ by as little as one nucleotide; such components could be assembled by using the complement of the polymorphic base to link otherwise identical blocks of sequence. In such an embodiment, the polymorphic nucleotide will form part of each 5′ flap released by ICR. ICR is sufficiently sensitive and accurate that an SNP can be determined using sequences that differ by only one nucleotide. Thus, the use of generic sequences can reduce assay costs and time to develop new assays. Further advantageously, ICR does not amplify target sequences, which reduces assay costs and contamination problems. ICR for target detection further provides the ability to offer an open architecture that can be adapted by each user for their specific needs. A user is supplied with materials including a universal detector (e.g., a “universal chip”) and generic oligonucleotides for ICR, where the generic oligonucleotides include identifier tags complementary to detection probes on the universal detector. With open architecture, the user adapts the methods and material provided herein to generate the target-specific component(s) needed for an assay, and uses the universal detector to measure results.

Advantageously, ICR can be carried out under isothermal conditions and provides an alternative to other methods of detecting target nucleotide sequences, including polymerase chain reaction (PCR) amplification of target sequences. ICR can easily be carried out in high-throughput mode. ICR assays are compatible with standard manual or automated detection systems. Because there is no need for special post-reaction containment, ICR can be run in reaction vessels such as standard microtiter plates. All reagents for one or a series of ICR assays can be combined in a single reaction mixture. The “hands-off” format, and the use of self-reporting readout methods such as electrochemical readout on a universal detector, or fluorescence resonance energy transfer (FRET) detection of probe cleavage, make ICR well suited to high-throughput use.

A description of ICR is found below. A description of the universal tag assay, including electrochemical readout, follows the disclosure of ICR.

Invasive Cleavage Reaction

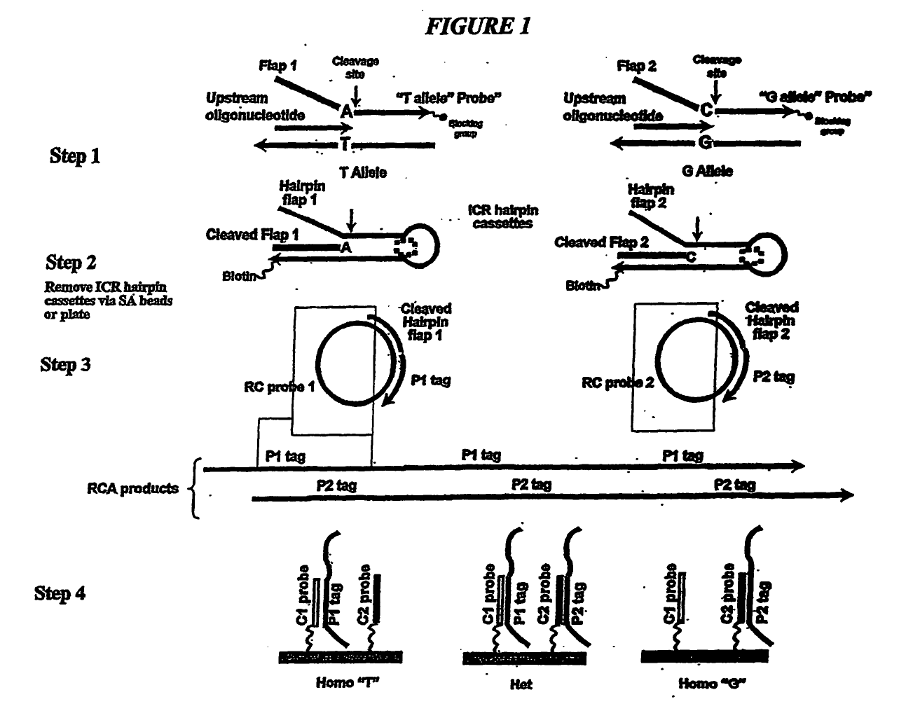

In accordance with one aspect of the present invention, a first invasive cleavage reaction (ICR) is carried out in a target-dependent manner. As illustrated in FIG. 1, Step 1, a first nucleic acid cleavage structure is formed on a template containing target nucleotide sequence. To form the cleavage structure, the sample being assayed is incubated with a probe and an upstream oligonucleotide as follows: a) the sample is incubated with an oligonucleotide probe that has a 3′ portion of sequence complementary to at least a portion the target nucleotide sequence of interest, and has a 5′ portion of sequence that is not complementary to target nucleotide sequence and does not bind to template; b) the sample is also incubated with an upstream oligonucleotide that is complementary to a portion of target nucleotide sequence and binds the template 5′ (upstream) of, and partially overlapping, the probe-complementary template sequence; c) both oligonucleotides hybridize to the template containing target nucleotide sequence, overlapping by at least one base pair to form a bifurcated structure; d) the bifurcated structure is recognized and cleaved by a structure-specific nuclease that cleaves the probe at a position one nucleotide 3′ (downstream) from the 3′ end of the upstream oligonucleotide; and e) the 5′ flap of the probe is released, containing the 5′ portion of the probe that was not complementary to target sequence and any overlapping nucleotide(s). Commonly, the probe and upstream oligonucleotide overlap by one nucleotide, such that the 5′ flap released by cleavage contains the unpaired probe 5′ sequence and the one overlapping nucleotide. The 5′ flap released by target-dependent ICR can be used as a reagent in subsequent reactions, or can be detected directly. The 3′ region of the probe, released by target-dependent ICR, can be ligated in subsequent reactions via the cleavage-created 5′ phosphate.

Advantageously, a single target can direct cleavage of multiple probes, as cleavage of the probe destabilizes the template-probe-upstream oligonucleotide complex and results in replacement of the cleaved probe with an uncut probe. In a given embodiment, probes are provided in excess and cleaved probes turn over without temperature cycling to produce many cleavage events per minute per target (linear amplification). ICR can be carried out at any suitable temperature; preferably, reactions are performed at temperatures near the melting temperature (Tₘ) of the probe, which favors rapid cycling of probe on and off the template (Kwiatkowski et al., Mol Diag 4:353-364; de Arruda et al., 2002, Expert Rev Mol Diagn 2:487-496). Preferably, reactions can be carried out at moderately elevated temperatures, e.g. between about 40° C. to about 75° C., using thermostable structure-specific endonucleases to cleave the bifurcated structure formed by the probe, the upstream oligonucleotide, and the template containing target nucleotide sequence.

It will be understood that, in accordance with the specificity of each probe and each upstream nucleotide for complementary sequence, a sample being assayed can be incubated with a plurality of probes and upstream oligonucleotides directed against a plurality of target nucleotide sequences, and a plurality of ICRs can occur in the same reaction mixture. It will also be understood that when using a plurality of probes to different regions in the same target nucleic acid or different target nucleic acids present in the same sample, along with their respective upstream oligonucleotides, the 5′ non-complementary regions of the probes can be different or the same.

In a preferred embodiment, as illustrated in FIG. 1, Step 1, ICR is used to determine which allele(s) of an SNP are present in a sample. A probe and upstream oligonucleotide must be complementary to the SNP allele(s) present in the sample being assayed in order to form a target-dependent cleavage structure and generate a cleavage product. The probe and upstream oligonucleotide are designed to bind to a portion of the template sequence flanking the SNP sufficient to identify the SNP with an acceptable level of confidence. The use of probe and upstream oligonucleotide pairs specific for each variant sequence, e.g., each allele of an SNP, provides a method of determining a plurality of alleles present in a sample. In the illustrative embodiment, an SNP is known to have a “T allele” and a “G allele”. The allele of the SNP may be determined by various methods. In one approach, the sample being assayed is contacted with sufficient amounts of probe and upstream oligonucleotide specific for the T allele, a cleavage structure is formed and cleaved, and a probe 5′ flap is released, where release of a 5′ flap indicates that at least one copy of the T allele was present in the sample. In such an embodiment, no 5′ flap release indicates the sample has no copy of the T allele. Alternately, the sample being assayed is contacted with a plurality of probes and upstream oligonucleotide pairs, where some pairs are specific for the T allele and some pairs are specific for the G allele, and the 5′ flap released from T-allele-specific probes is distinguishable from the 5′ flap released from G-allele-specific probes. One of skill in the art would understand that the 5′ flap released by target-dependent ICR does not contain the target nucleotide sequence but rather, contains non-target sequence chosen to serve as an indicator of the target nucleotide sequence. When the polymorphic nucleotide of an SNP is being determined, it will be understood that the target nucleotide sequence includes as much of the surrounding nucleotide sequence as is necessary to identify the SNP to the desired level of accuracy. Thus, a probe 5′ flap containing the complement of the polymorphic nucleotide of a SNP is not considered to contain a copy of target nucleotide sequence, as it does not contain any of the flanking sequence used to identify the SNP.

In one preferred embodiment, the 5′ flap released by target-dependent ICR can be detected directly. The 5′ flap may be detectably labelled, e.g., with a detectable fluorescent, colorimetric, or radioactive label, or with an addressable ligand such as biotin, an enzyme, or an antibody. Advantageously, the 5′ flap may be part of a signal complex such that cleaving the probe to release the 5′ flap alters a detectable signal, for example as in the FRET signalling system disclosed by Stevens et al., (2001, Nuc Acids Res 29:e77) or Kwiatkowski et al. (1999, Mol Diag 4:353-364). The 5′ flap may be detected by electrophoresis, enzyme-linked immunosorbent assay (ELISA) of labelled flaps, matrix-assisted laser desorption/ionization time-of-flight (MALDI-TOF) mass spectrometry, and other methods as may be determined by one of skill in the art.

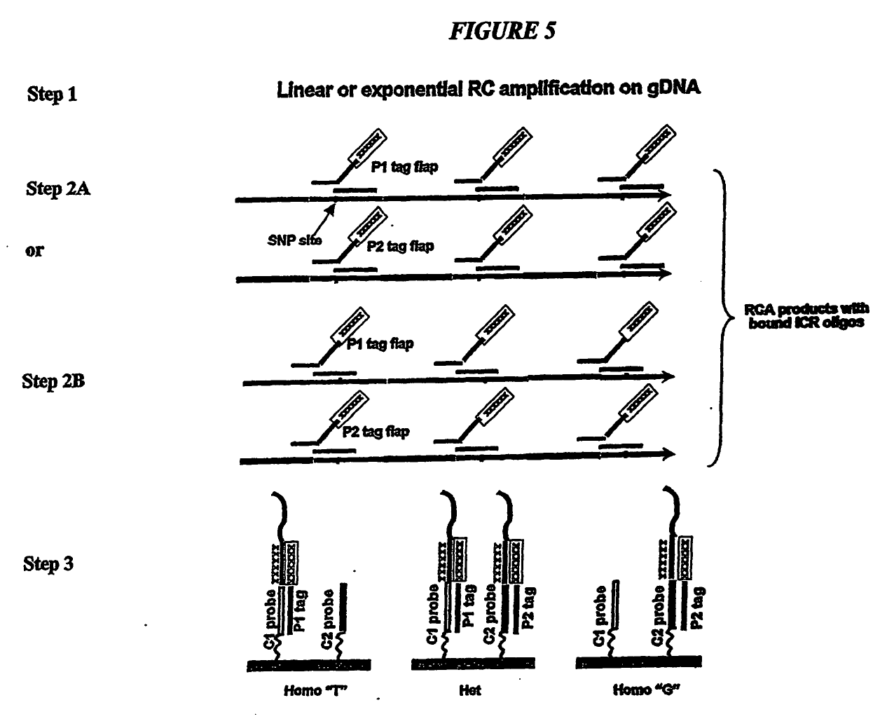

In accordance with one aspect of the present invention, the probe 5′ flap released from a first ICR contains an identifier tag chosen to serve as an identifier for the target nucleotide sequence. Such a 5′ flap is a tagged molecule suitable for use in a universal tag assay as disclosed in U.S. Provisional Application 60/424,656 filed Nov. 6, 2002, the disclosure of which is incorporated by reference herein in its entirety. In one embodiment, a target nucleotide sequence is detected by using target-dependent ICR to generate at least one tagged molecule having an identifier tag corresponding to that target, incubating the tagged molecule(s) with a universal detector having at least one detection probe complementary to the identifier tag, and measuring hybridization of the identifier tag to its complementary detection probe, where such hybridization indicates the presence of the target in the sample being assayed. FIG. 5 illustrates an embodiment in which target-dependent ICR is carried out on a template containing multiple copies of a target nucleotide sequence having an SNP, tagged 5′ flaps are released after cleavage, and hybridization of tagged 5′ flaps to specific detection probes indicate which allele(s) of the SNP are present in the template.

It will be understood that the specificity of ICR provides that a sample can be incubated with a plurality of probes and upstream oligonucleotides directed against a plurality of target nucleotide sequences, such that a plurality of ICRs can occur in the same reaction mixture and a plurality of tagged molecules can be generated and measured in the same reaction mixture. Preferably, a universal detector contains at least one detection probe complementary to each identifier tag in the plurality of tagged molecules generated by ICR. Hybridization of each identifier tag to a complementary detection probe on the universal detector can be measured, where hybridization indicates the presence of the corresponding target in the sample assayed.

Serial ICR

In accordance with another aspect of the present invention, the 5′ flap released by target-dependent ICR can be used in subsequent reactions. In a preferred embodiment, more than one ICR reaction is carried out, more preferably in a linked series, e.g. as illustrated in FIGS. 1, 6, 8, and 9. Advantageously, serial ICR is suitable to detect small amounts of targets in a sample, detecting fewer than 1000 copies of target nucleotide sequence with no prior target amplification, or discriminating SNPs of a gene using as little as 20 ng of human genomic DNA (Hall et al., 2000, Proc Natl Acad Sci 97:8272-8277). Typically, 10³ 5′ flaps can be released in one hour by a single ICR, and two ICR in series can yield 10⁶-fold amplification of the 5′ flap signal. Serial ICR is sometimes referred to as “ICR²” or “ICR-squared” to reflect the additional amplification of signal by ICR in series. Each subsequent ICR can provide any of a variety of functions including signal amplification, additional levels of stringency, quality control, or transduction of signals from one form to another. Signal transduction occurs when target nucleotide sequence in a sample generates an unlabelled cleavage product from one ICR, and the cleavage product participates in another ICR that generates a tagged molecule that can be detected by electrochemical means.

In serial ICR, a second ICR is carried out using the cleavage product released from the first ICR, wherein the cleavage product is the probe 5′ flap cleaved at a position one nucleotide 3′ of the overlapping sequence. The first ICR is usually, but not always, a target-dependent ICR. In serial ICR, a first target-dependent ICR advantageously provides a discrimination function, by providing a cleavage product that is released only when the corresponding target sequence is present. The cleavage product of a second ICR can be detected directly as described above, or used in subsequent reactions. Preferably, the 5′ flap released from a first ICR is used as an upstream oligonucleotide in a second ICR, as illustrated in FIGS. 1, 6, 8, and 9. More preferably, a second ICR is carried out using a “hairpin cassette” containing a probe and a target coupled through a hairpin turn, as illustrated in FIG. 1. The target and probe sequences of the hairpin cassette are paired through their complementary portions, and their unpaired portions provide a target for the upstream oligonucleotide, a probe's 5′ flap. The target in a second ICR is usually, but not necessarily, a synthetic sequence designed to be complementary to a specific probe 5′ flap generated in a first ICR.

A second ICR can provide an additional discrimination step as illustrated in FIG. 1 Step 1, where at least two different types of hairpin cassettes corresponding to each allele are used. In such an embodiment, a hairpin cassette of the second ICR is allele-specific in that the hairpin cassette is specific for the cleavage product of the first ICR specific for that allele. The 5′ flap released from the first target-dependent ICR is used as an upstream nucleotide in a second ICR, binding complementary target sequence and overlapping the probe to form a bifurcated cleavage structure on the hairpin cassette. Preferably, uncleaved hairpin cassettes are removed to preclude competition with the sequences in subsequent steps. Hairpin cassettes can be addressably labelled and removed from the reaction mixture after ICR Addressable labels include ligands such as biotin, and uncleaved hairpin cassettes can be removed using ligand-binding molecules coupled to a solid support, e.g., streptavidin (SA) coated beads to removed biotin-labelled cassettes. Preferably, the hairpin cassette is 3′ labelled with biotin, and removed by capture to a SA-coated surface. Alternately, hairpin cassettes are not removed and the reagent that binds the 5′ flap of the probe in a subsequent step is added in excess, to provide sufficient reagent for the cleaved 5′ flaps to bind and drive the subsequent reaction. In this embodiment, the 5′ flaps still attached to uncleaved hairpin cassettes also bind to the reagent, but this structure does not generate spurious signals because it cannot drive the subsequent reaction.

Template for ICR

As used herein, “template” refers to all or part of a polynucleotide containing at least one target nucleotide sequence. Template suitable for ICR includes DNA and RNA. Template DNA includes but is not limited to genomic DNA (gDNA), cDNA, PCR product, ligation (LCR, OLA) product, Rolling Circle (RC) amplification product. Template RNA includes but is not limited to mRNA, tRNA, rRNA, siRNA, and viral DNA, and ICR probes that bind to them can be cleaved by suitable enzymes (Eis et al. 2001, Nat Biotechnol 19:673-676). Synthesized DNA or RNA sequences are suitable templates, as are nucleotide analogues and modified nucleotides such as PNA or LNA. ICR requires single stranded template; if desired, single stranded template can be generated from double-stranded structures by thermal denaturation, digestion, or local unwinding of paired strands. One of skill in the art can determine the suitability of a template empirically by attempting to carry out ICR and determining whether a product is generated, e.g., by testing for a known control sequence.

Use of Amplification Products as Template for ICR

In accordance with one aspect of the present invention, amplification of at least a portion of a polynucleotide containing target nucleotide sequence provides amplification products suitable as a template for ICR. A polynucleotide containing target nucleotide sequence can be amplified by methods including, but not limited to, rolling circle (RC) amplification, polymerase chain reaction (PCR) amplification, ligase chain reaction (LCR) amplification, oligonucleotide ligation amplification (OLA), self-sustained synthetic reaction (3SR/NASBA), and Q-β replicase (Q-beta or Qβ) amplification. Double stranded amplification products are denatured to provide single stranded template for ICR; products may be completely denatured by separating them into single strands, or may be locally denatured, e.g., by “unwinding” double stranded product to generate single stranded template in the region of interest. It will be understood that denaturation, in particular localized denaturation, can be achieved at a range of temperatures for some sequences.

RC amplification products as template. The product of RC amplification of a target nucleotide sequence provides a suitable template for ICR. By way of example, RC amplification can be carried out on genomic DNA using methods well known in the art, for example as disclosed by Kool et al. (U.S. Pat. Nos. 5,714,320, 6,368,802 and 6,096,880), Landegren et al. (U.S. Pat. No. 5,871,921), Zhang et al. (U.S. Pat. Nos. 5,876,924 and 5,942,391) and Lizardi et al. (Lizardi et al., 1998, Nature Genet 19: 225-232, and U.S. Pat. Nos. 5,854,033, 6,124,120, 6,143,495, 6,183,960, 6,210,884, 6,280,949, 6,287,824, and 6,344,329), and as described in U.S. patent application Ser. No. 10/138,067, and U.S. Provisional Applications 60/404,195 and 60/424,656, the entire contents of each of which are hereby incorporated by reference. Advantageously, RC amplification can be carried out as an isothermal amplification method having high specificity and sensitivity for target nucleotide sequences and a low level of nonspecific background signal. Further advantageously, target-dependent ligation of the circular probe used in RC amplification can be used to discriminate among variant sequences, for example to carry out allelic discrimination or determine single nucleotide polymorphisms (SNPs), splice variants, mutants, or alleles of a nucleotide sequence. Linear RC amplification generates a linear, single-stranded product, while exponential RC amplification (also known as “hyperbranched” RC amplification) using secondary primers generates a branched, double-stranded product.

As illustrated in FIG. 5, ICR can be used to detect target nucleotide sequences in an RC amplification product. ICR is carried out using probes and upstream oligonucleotides specific for the targets being assayed. It will be understood that due to the specificity of binding of each probe and each upstream nucleotide for complementary sequence, a sample being assayed can be incubated with a plurality of probes and upstream oligonucleotides directed against a plurality of target nucleotide sequences, and a plurality of ICRs can occur in the same reaction mixture. When ICR is performed on amplification product, a probe and upstream oligonucleotide set may be directed entirely to regions of target nucleotide, or may span regions of target and generic sequence in the RC amplification product. In the former case, both the probe and the upstream oligonucleotide are complementary to target nucleotide sequence. In the latter case, either the template-binding portion of the probe, or the upstream oligonucleotide, can be complementary to generic sequence, as long as one of the components binds to at least a portion of a target-distinguishing sequence, and both components bind to template sequences that overlap by at least one nucleotide to form a cleavage structure.

It will be understood that one of skill in the art can determine the assay design best suited to a particular embodiment. In the illustrative embodiment shown in FIG. 5, one ICR reaction may be carried out to generate a tagged molecule suitable for use in a universal tag assay. Alternately, more than one ICR, preferably serial ICR, can be carried out to generate a tagged molecule suitable for use in a universal tag assay. To determine the identity of the polymorphic nucleotide(s) in an SNP, a region of genomic DNA from a diploid organism is amplified by RC amplification using at least one, preferably two or more, circular probes specific for alleles of the SNP. When two probes are used, the RC amplification product could contain copies of: 1) only the T allele corresponding (homozygous T); 2) only the G allele (homozygous G); or 3) both the T and the G allele (heterozygous). As illustrated in FIG. 5 Steps 2A or 2B, template generated from a heterozygote would generate both P1 and P2 identifier tags, each of which would bind to its complementary detection probe C1 and C2 respectively and generate a signal. Likewise, template generated from an organism homozygous for the T allele would only generate P1 tag which would produce a signal upon binding to the C1 detection probe, and template generated from an organism homozygous for the G allele would only generate P1 tag which would produce a signal upon binding to the C1 detection probe.

PCR amplification products as template. The product of PCR amplification of a target nucleotide sequence provides a suitable template for ICR. The polymerase chain reaction (PCR), as described by Mullis et al. in U.S. Pat. Nos. 4,683,195 and 4,683,202 (the entire contents of each of which are hereby incorporated by reference), describe a method for increasing the concentration of a segment of target sequence in a mixture of genomic DNA without cloning or purification. Advantageously, PCR can be used to directly increase the concentration of the target to an easily detectable level. This process for amplifying the polynucleotide containing target sequence involves introducing a molar excess of two oligonucleotide primers complementary to their respective strands of the double-stranded target sequence to the DNA mixture containing the desired target sequence. The mixture is denatured and then allowed to hybridize. After hybridization, the primers are extended with polymerase so as to form complementary strands. The steps of denaturation, hybridization, and polymerase extension can be repeated as often as needed, in order to obtain relatively high concentrations of a segment of the desired target sequence. Double stranded PCR amplification product is denatured to provide single stranded template to carry out ICR.

Enhancing ICR Signals

Optionally, the signal generated by the tagged molecule can be enhanced with a “development” reagent. As illustrated in FIGS. 5, 6, and 7, a tagged molecule may contain not only an identifier tag but also a sequence or other chemical moiety for binding a development reagent that enhances, stabilizes, or amplifies the signal generated by the binding of an identifier tag to a complementary detection probe. Preferably the development reagent enhances, stabilizes, amplifies, or otherwise optimizes electrochemical detection of binding to identifier tags to detection probes. A development reagent may be a short oligonucleotide in a tagged molecule, the complement of which is found in the corresponding detection probe. Alternately, the corresponding detection probe does not have the complement of the development reagent and the development reagent is added to a reaction mixture, e.g., after contacting tagged molecules with a universal detector and allowing identifier tags to bind to detection probes. In some embodiments, the interaction of the 5′ region of the 5′ flap and an added development reagent could be via an antigen-antibody interaction. The sequence of the development reagent (and its complement) may be generic or unique. A generic development reagent is found on all tagged molecules in an assay and is likely to stabilize the interaction between identifier tag and detection probe, or to enhance the signal generated thereby. A unique development reagent can be used to enhance differentiation of signals generated by distinct identifier tags binding to complementary detection probes on a universal detector. Alternately, signal discrimination may be enhanced by using development reagents only with the tagged molecules corresponding to certain targets, and not using development reagents with the tagged molecules corresponding to other targets.

Enzymes for ICR

Binding of a probe and an upstream oligonucleotide to a target creates a bifurcated overlapping complex known as a cleavage structure, and structure-specific 5′-nucleases cut the cleavage structure at a position 3′ to the site of the overlap, releasing the 5′ flap and any probe nucleotide that was base-paired in the overlap. Preferably, enzymes suitable for ICR in accordance with the present invention have high specificity for the cleavage structure, a rapid turnover rate, thermostability, and the ability to recognize either DNA or RNA templates. Enzymes used for ICR fall generally into two classes: those derived from the 5′ exonuclease domains of DNA polymerase I of thermophilic eubacteria (Pol I-type enzymes) and flap endonuclease (FEN-1)-type enzymes, typically isolated from thermophilic archaebacteria, where the enzymes do not have associated DNA polymerase activity (Kwiatkowski et al., 1999, Mol Diag 4:353-364 and Kaiser et al., 1999, Proc Natl Acad Sci USA 274:21387-21394, the entire contents of each of which are hereby incorporated by reference). Enzymes of the Pol I-type have been altered to eliminate their polymerase activity, providing altered or mutant enzymes that act predominantly as 5′ nucleases that are capable of cleaving nucleic acids in a structure-specific manner in the absence of interfering synthetic activity, for example as disclosed in U.S. Pat. Nos. 5,795,763, 5,614,402, and 5,541,311, the entire contents of each of which are hereby incorporated by reference.

Where the polymerase structure is altered, it is not intended that the invention be limited by the means by which the structure is altered. In one embodiment, the structure of the polymerase is altered by changing a single nucleotide in the DNA encoding the polymerase. In another embodiment, the structure of the polymerase is altered by deleting one or more nucleotides in the DNA encoding the polymerase. In yet another embodiment, the structure of the polymerase is altered by inserting one or more nucleotides into the DNA sequence encoding the polymerase. It is contemplated that the change in DNA sequence may manifest itself as change in amino acid sequence. Alternately, native polymerase activity may be inhibited by synthesis inhibitors.

The present invention contemplates structure-specific nucleases from a variety of sources, including mesophilic, psychrophilic, thermophilic, and hyperthermophilic organisms. Thermostable structure-specific nucleases are contemplated as particularly useful in that they operate at temperatures where nucleic acid hybridization is extremely specific. Advantageously, hybridization at higher temperatures favors stringent hybridization such that probes can distinguish between single-base differences between various target nucleotide sequences in a sample. In a preferred embodiment, thermostable structure-specific nucleases are 5′ nucleases of the Pol I type selected from the group consisting of altered polymerases derived from the native polymerases of species of the genus Thermus, including, but not limited to Thermus aquaticus (TaqPol), Thermus thermophilus (TthPol), and Thermus flavus. It should be understood that the invention is not limited to the use of thermostable 5′ nucleases. Thermostable structure-specific nucleases from the FEN-1, RAD2 and XPG class of nucleases are also preferred, including FEN-1-type enzymes isolated from the archaebacteria Archaeoglobus fulgidus (AfuFEN), Pyrococcus furiosus (PfuFEN), Methanococcus jannaschii (MjaFEN), and Methanobacterium thermoautotrophicum (MthFEN). AfuFEN is particular preferred for the robustness of its activity, combining an acceptably high turnover rate with high substrate specificity.

Enzymes suitable for use in the present invention include enzymes that exhibit little or no cleavage activity when substrate and probe are present but the upstream oligonucleotide is absent (Kwiatkowski et al., 1999, Mol Diag 4:353-364). Advantageously, the ability of these enzymes to discriminate invasive structures (e.g., probe and upstream oligonucleotide bound to template, forming a bifurcated cleavage structure) from noninvasive structures (e.g., probe bound to template in the absence of upstream oligonucleotide), provides sensitivity and specificity in each ICR reaction.

For ICR on RNA templates, the 5′ nuclease must recognize cleavage structures formed upon an RNA template. Advantageously, eubacterial 5′ nucleases exhibit activity on such substrates.

ICR Assay: Reaction Conditions

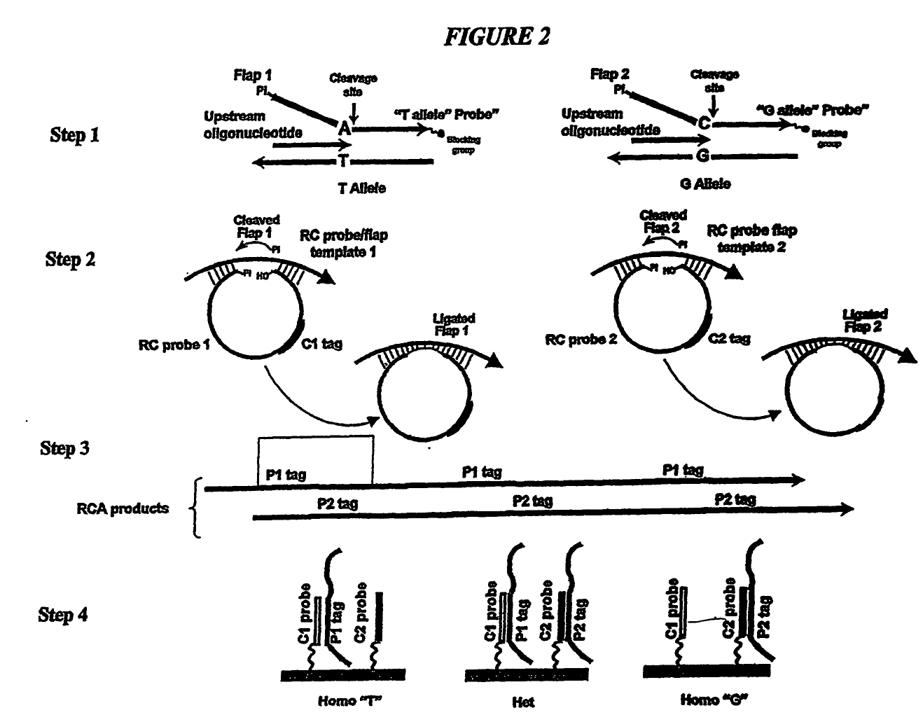

The optimal temperature for isothermal signal amplification, or probe cycling (cleavage of multiple probes for each target molecule being detected) is influenced by the Tₘ of the probe, which is dependent on oligonucleotide length, base composition, oligonucleotide concentration, salt and magnesium concentration, and type of enzyme. The optimal temperature for a particular reaction is influenced by both the probe(s) and upstream oligonucleotide(s) used in an assay. Generally, optimal reaction temperatures range from approximately 30° C. to approximately 80° C., more preferably from approximately 40° C. to approximately 75° C. Preferably, more sensitive DNA detection assays have been optimized to run at approximately 65° C. One of skill in the art can determine the optimal temperature for a particular embodiment, e.g., as in the temperature optimization curve shown in Kwiatkowski et al., 1999 (Mol Diag 4:353-364, FIG. 2) showing activity of various enzymes tested over a range of temperatures.

Use of 5 ′ Flap in Reactions Other Than ICR

In accordance with another aspect of the present invention, the 5′ flap released from the probe by target-dependent ICR can be used in subsequent reactions other than ICR. Subsequent reactions can provide signal amplification, additional levels of stringency, quality control, signal transduction, and other advantageous functions.

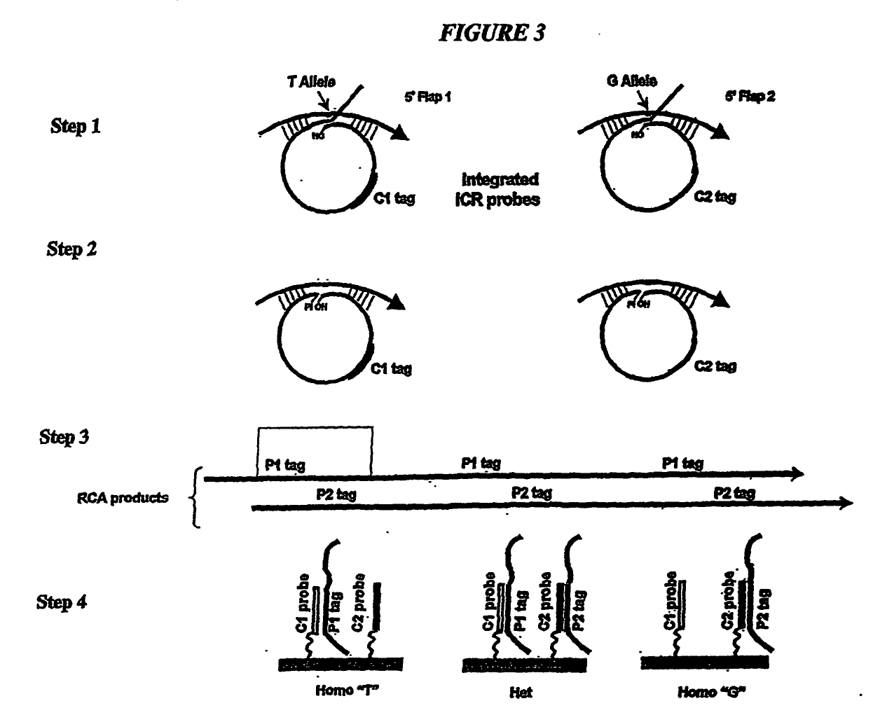

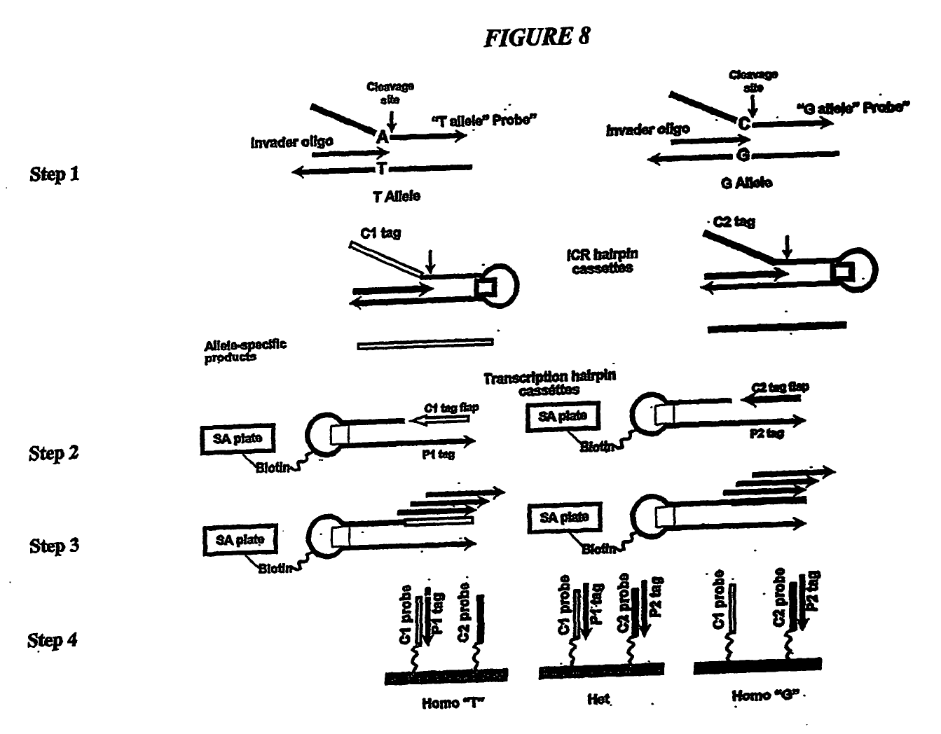

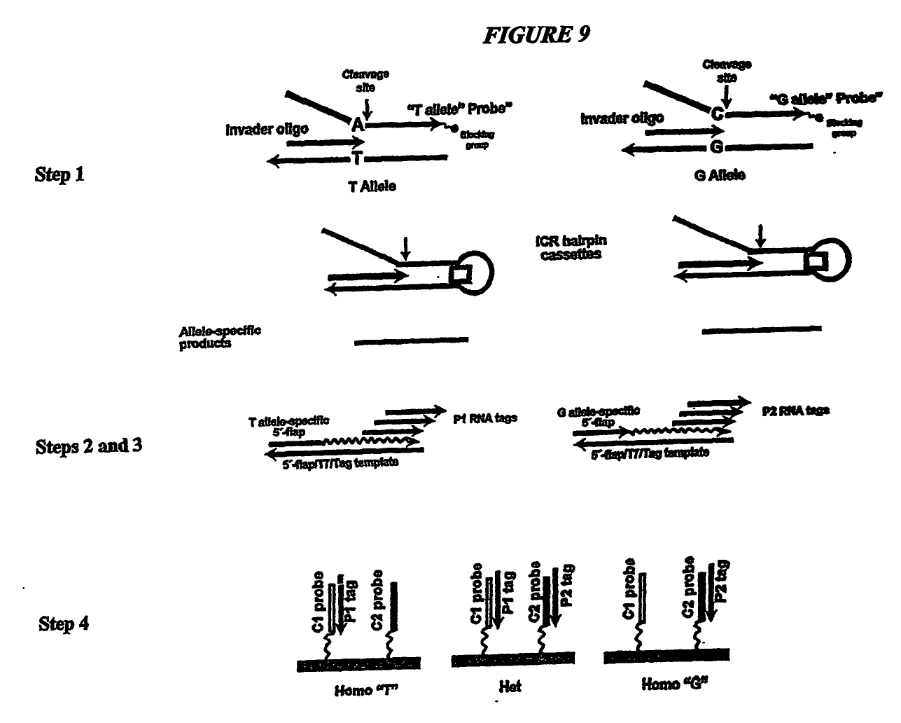

In accordance with one aspect of the present invention, the 5′ flap released by ICR can be used in a ligation reaction, for example, wherein DNA probes specific for a particular 5′ flap hybridize to contiguous portions of the 5′ flap and are ligated, and further wherein the probes introduce exogenous sequence not found in the 5′ flap. In one embodiment, the cleaved 5′ flap is ligated into a rolling circle probe, to generate a circular molecule which can be used in a rolling circle amplification procedure (FIG. 2). One of skill in the art would also understand that the RC probe/flap template (FIG. 2) could be used as the immobilized detection probe and the 3′ terminus of the detection probe can be extended to detect the presence of the target in the sample. In another embodiment, cleavage of an integrated ICR probe and subsequent ligation generates a circular molecule which can be used in a rolling circle amplification procedure (FIG. 3). In some embodiments, the 5′ flap hybridizes and is ligated to a hairpin structure that contains an RNA polymerase promoter binding site and an identifier tag as shown in FIG. 8. Alternatively, the 5′ flap may be ligated into a rolling circle probe to generate a circular molecule (FIG. 2). The rolling circle probe may be hybridized to a nucleic acid referred to herein as the rolling circle probe/flap template. The rolling circle probe/flap template comprises nucleotide sequences complementary to the flap and nucleotide sequences complementary to the terminal regions of the rolling circle probe. It will be appreciated that the length of the terminal regions of the rolling circle probe to which the rolling circleprobe/flap template is complementary may be any length which allows the rolling circle probe/flap template to hybridize to the rolling circle probe. The rolling circle probe/flap template may also comprise a promoter from which an RNA polymerase may initiate transcription, or alternatively, the rolling circle probe/flap template may be extended with a polymerase across a nucleotide sequence in the rolling circle probe which is complementary to a promoter from which RNA polymerase may initiate transcription such that the extension product contains the promoter. Preferably, the RNA polymerase is T7. Transcription of the ligation product by RNA polymerase generates an RNA molecule containing an identifier tag. Such an RNA molecule is a tagged molecule suitable for use in a universal tag assay. In another embodiment, the cleaved 5′ flap hybridizes to a template and is extended with a polymerase. The extended flap contains an RNA polymerase binding site and an identifier tag. Addition of the RNA polymerase results in transcription of an RNA molecule containing an identifier tag (FIG. 9).

In accordance with another aspect of the present invention, the 5′ flap released by ICR can be used as a polymerization primer for rolling circle (RC) amplification of a circular oligonucleotide or “RC probe” to generate tagged molecules for use in a universal tag assay. As illustrated in FIG. 1 Step 3, the cleavage product of the second ICR in a serial ICR is used as a primer for RC amplification. In one preferred embodiment, preformed circular RC probes are used, and each RC probe amplified by this method contains the complement of an identifier tag chosen to serve as a distinct identifier for a particular target. As illustrated in FIG. 1, Step 3, RC probe primed by 5′ flap generated when the T allele is present contains the complement of the P1 tag, and the RC probe specific for the 5′ flap generated when the G allele is present contains the complement of the P2 tag. RC amplification primed by a 5′ flap generates a multimeric product containing identifier tags capable of binding to at least one complementary detection probe on a universal detector. The P1 tag binds to the C1 detection probe, and the P2 tag binds to the C2 detection probe. Measurement of identifier tag binding to the C1 probe indicates that the T allele was present in the sample. Measurement of identifier tag binding to the C2 probe indicates that the G allele was present in the sample. Measurement of identifier tag binding to both C1 and C2 probes indicates that both the T allele and the G allele were present in the sample. No binding to the C1 tag indicates that the G allele was not present in the sample, e.g., the sample was from a diploid organism homozygous for the T allele; no binding to the C2 tag indicates that the T allele was not present in the sample, e.g., the sample was from a diploid organism homozygous for the G allele. It will be understood that, if desired, one of skill in the art may practice any such assay with suitable controls to demonstrate that positive results (measuring binding of tags to probes) and negative results (not measuring binding of tags to probes) are reliable results.

In another embodiment, only one ICR is performed prior to an RC amplification step to generate tagged molecules. In this version, the probe's 5′-flap would become the RC polymerization primer upon cleavage. Optionally, the 3′ end of the probe is modified with a chemical moiety such as biotin, and captured using SA, to preclude competition between uncleaved probes and cleaved 5′ flaps for the primer-binding site on the RC probe. Alternately, competition is prevented by adding “quencher” oligonucleotides in excess of the target-specific probes, e.g., as disclosed by Eis et al. (2001, Nat Biotechnol 19:673-676) using “arrestor” oligonucleotides. In such an embodiment, the quencher oligonucleotide would preferably be fully complementary to the target-specific region of the probe and only partially complementary to the 5 ′-flap.

In another preferred embodiment, each 5′ flap contains an identifier tag. When the 5′ flap is used as a polymerization primer, binding of the primer to the circular probe provides an additional discrimination step, and RC amplification of the circular probe amplifies the identifier tag sequence.

In yet another preferred embodiment, 5′ flaps are mixed with RC probes in linear form, where the linear molecules have 3′ and 5′ sequence complementary to the 5′ flap. Circular RC probes will hybridize to complementary 5′ flaps and be ligated. In one embodiment, the 5′ flap also serves as a polymerization primer for the circularized RC probes. Thus, the 5′ flap released by ICR can participate in additional discrimination steps that enhance the accuracy of the assay and decrease the potential for spurious signals.

Trimming amplification products. Optionally, a multimeric RC amplification product can be cleaved or “trimmed” to release shorter tagged molecules. The shorter tagged molecules may optionally contain other sequences complementary to the circular probe. Cleavage of RC amplification products is described in detail in U.S. patent application Ser. No. 10/138,067, and U.S. Provisional Applications 60/404,195 and 60/424,656, and references cited therein, the entire contents of each of which is hereby incorporated by reference. Briefly, a circular probe is designed to include sequences that will provide cleavage sites in the complementary RC amplification product. In one embodiment, the amplification product may contain self-complementary sequences that form hairpin structures that contain at least one restriction enzyme recognition site for a restriction enzyme involved in the trimming step. Suitable restriction enzymes include Type II restriction enzymes such as EcoRI, or Type US restriction enzymes such as FokI.

In another embodiment, the amplification product may include sequences involved in trimming the amplification product by restriction enzymes, where the amplification product encodes one strand of the restriction enzyme recognition site, and the double-stranded restriction enzyme recognition site is formed upon addition of at least one auxiliary oligonucleotide. As used herein, an “auxiliary oligonucleotide” is an oligonucleotide, preferably DNA or RNA, that can be used to create a region of double-stranded DNA or RNA, or DNA/RNA heteroduplex, to generate a double stranded restriction digestion site that permits cleavage of the single-stranded polynucleotide to generate a smaller tagged molecule. Suitable restriction enzymes include Type II restriction enzymes such as EcoRI, or Type IIS restriction enzymes such as FokI.

If RNA amplification products are generated, the amplification products may encode autocatalytic structures such as ribozymes as disclosed by Kool et al., U.S. Pat. Nos. 6,096,880 and 6,368,803, the entire contents of each of which are hereby incorporated by reference. In another embodiment, a tagged RNA molecule is generated by transcription of a ligation product containing a T7 promoter hairpin oligonucleotide and the complement of an identifier tag.

As provided herein, RC amplification primed by a 5′ flap released from ICR provides multiple advantages including but not limited to: 1) signal amplification, where a single 5′ flap molecule primes the reaction to generate hundreds, thousands, or millions of copies of the circular probe containing the detectable identifier tag; 2) additional levels of stringency; quality control, because RC amplification only occurs if the previous ICR reaction(s) generated the 5′ flap to provide the polymerization primer specific for a particular circular RC probe; and 4) signal transduction, where recognition of a target nucleotide sequence in the sample generated an unlabelled cleavage product (5′ flap) that either directly, or through a cascade of subsequent reactions, generated an RC amplification product containing a detectable signal, namely an identifier tag complementary to at least one detection probe in a universal detector.

Assays using ICR do not amplify the target of interest but rather, generate and amplify an unrelated signal only in the presence of the correct target sequence. By avoiding target amplification, ICR avoids the possibility of generating incorrect amplification products that can carry over into other steps of the assay and generate spurious results. Because of the degree of sensitivity and discrimination of invasive cleavage assays, these assays can be used to detect subattomole levels of target nucleic acids within complex mixtures, and variant sequences such as single nucleotide polymorphisms (SNPs) can be detected directly from genomic DNA without the need for prior amplification of the target sequence. (Hall et al., 2000, Proc Natl Acad Sci USA 97:8272-8277; Stevens et al., 2001, Nuc Acids Res 29:e77).

It will be understood by one of skill in the art that ICR is a versatile method that can, alone or in combination with other methods, be used to generate tagged molecules suitable for use in a universal tag assay. Preferably, ICR can be used to generate tagged molecules for use with a universal detector such as a “universal chip” where ICR provides a desirable combination of great sensitivity and accuracy, with ease of assay design. The ability to use non-target-complementary 5′ flaps generated by ICR as signals affords a simple but accurate means for converting a target-specific discrimination event into multiple, non-target-specific events. In one preferred embodiment, the one base pair (1 bp) difference found in an SNP is converted into, e.g., a 15 base pair identifier tag. More particularly, the information contained in the 1 bp difference in the polymorphic nucleotide of an SNP is detected by target-dependent ICR with great accuracy and sensitivity. The 5′ flap released from the first ICR carries the information content of the SNP, i.e., the identity of the polymorphic nucleotide, where the greater complexity of the 5′ flap has already increased the information content and potential for accuracy of the assay. The 5′ flap carrying the information content of the SNP may participate in subsequent reactions that not only preserve the information content of the SNP but may also “test” that information content at increasing levels of stringency, and/or may amplify the signal to facilitate or enhance accurate detection. Further, the 5′ flaps released from ICR possess a 3′-OH group, which enables the 5′ flaps to be used as primers in polymerase reactions, or as one component of a ligation reaction wherein the second component contains a 5′ phosphate, which enables the two components to be ligated. It is also understood by one of skill in the art that the released 3′ target-specific region of the ICR probe possesses a 5′ phosphate, which can be used in a subsequent ligation reaction.

Further advantageously, uncleaved probes or hairpin cassettes as disclosed herein can be removed from the reaction mixture, thereby minimizing the possibility of generating spurious signals from the 5′ flap regions of uncleaved probes. In some embodiments, uncleaved probes and hairpin cassettes can be physically removed, e.g., by digestion with exonuclease or by use of a biotin-SA interaction to capture probes and cassettes 3′-labelled with biotin. In other embodiments, uncleaved probes can be “titrated out” of a reaction, e.g., using quencher oligonucleotides that bind to the target-specific region of the probe and to a portion of the 5′ flap region, specifically preventing the 5′ flap region of the uncleaved probe from interacting with another template (Eis et al., 2002, Nat Biotechnol 19:673-676). Uncleaved probes and hairpin cassettes can also be excluded from a subsequent reaction steps by design, e.g. where the 5′ flap portion of an uncleaved probe or cassette cannot stably interact with the template to which the cleaved 5′ flap binds to drive a subsequent step.

Universal Tag Assay

The term “target” as used herein refers to a molecule such as a polynucleotide, polypeptide, small organic molecule, or other molecule of interest in a particular application. In a particular embodiment, the term “target” may be a “target nucleotide sequence” which is a nucleotide sequence of interest in a particular application. One of skill would understand that a target nucleotide sequence for any particular application is a nucleotide sequence containing sufficient information to identify the target of interest in a particular application. In an embodiment in which multiple targets are being assayed, target nucleotide sequences for each of the multiple targets may be the same or different lengths. Generally, the target nucleotide sequence is a portion of the nucleotide sequence of a target polynucleotide. One of skill in the art would understand that the universal tag assay of the present invention detects the presence of a target polynucleotide in a sample by detecting the target nucleotide sequence contained therein.

The term “target nucleotide sequence” may also refer to information encoded by a polynucleotide, e.g., motifs, domains, secondary structure of the polynucleotide, hybridization characteristics of a polynucleotide, amino acid sequence of a polypeptide, secondary or tertiary structure of a polypeptide, physical properties of a polypeptide, ligand binding sites of a polypeptide, information about homology or phylogeny in nucleotide or amino acid sequence, and any other information useful in a particular application.

When methods disclosed herein are used to determine an SNP, it is understood that a target nucleotide sequence encompasses a nucleotide sequence with sufficient information content to reliably identify the polymorphic nucleotide(s) of the SNP in a sample. In one embodiment, a target nucleotide sequence that is ten (10) nucleotides in length may be sufficiently distinctive (have sufficient information content) to reliably target probes to the SNP of interest, in a sample containing multiple copies of the entire genome of an organism. In other embodiments, the SNP may be located in a gene that is present in multiple copies such that similar stretches of sequence are found throughout the genome. In such a case, the target nucleotide sequence for that SNP will have to be longer (have higher information content) in order to reliably target to the SNP of interest. The “target” of the assay may be variously defined as the SNP of interest (in which case the target is one nucleotide in length), or the gene having the SNP of interest, or the individual having a particular diploid SNP genotype for that gene, or the individual having the phenotype associated with the SNP of interest, u.s.w. Regardless of how the target is defmed, one of skill in the art would understand that a target nucleotide sequence for an SNP will be determined by the information content necessary to reliably identify the SNP in a sample to at least a minimum desired level of accuracy. A target nucleotide sequence may contain additional information beyond the minimum required. One of skill in the art can select a target nucleotide sequence for an SNP of interest based on the nucleotide sequence of the region surrounding the SNP.

A “tagged molecule” contains an identifier tag for a particular target. A tagged molecule may contain additional sequence. A tagged molecule is a molecule that interacts with the universal detector as follows: the tagged molecule containing an identifier tag is incubated with the universal detector having detection probes, and the identifier tag in the tagged molecule hybridizes to a complementary detection probe of the universal detector. Tagged molecules are generated by one or more steps including at least one target-dependent step. Thus, a tagged molecule containing an identifier tag is generated only when the target corresponding to that identifier tag is present in the sample. Preferably, an identifier tag is an oligonucleotide having a known nucleotide sequence. Because an identifier tag is generated only when the corresponding target is present in a sample being assayed, a tagged molecule containing only an identifier tag is sufficient to indicate the presence of the corresponding target in the sample being assayed.

Identifier tags in tagged molecules suitable for use with a universal detector of the universal tag assay may be DNA or RNA oligonucleotides, and may include modified bases, non-naturally-occurring bases, and may further include labels, ligands, or other materials and modifications suitable to a particular application. Generally, tagged molecules are oligonucleotides or polynucleotides (depending on length). Advantageously, the use of identifier tags and a universal detector having complementary detection probes provides a universal tag assay that is independent of the organism or tissue being analyzed. Multiple target nucleotide sequences can be detected simultaneously, due to the one-to-one correspondence between each identifier tag and the target nucleotide sequence for which it serves as an identifier, and further due to the specificity of hybridization of each identifier tag to its detection probe.

Tags and Probes

One aspect of the invention provides a set of tags and probes for use in accordance with the methods and compositions herein disclosed. Detection probes used with universal detectors of the present invention are directed to complementary tags that serve as identifiers for targets in a sample. Likewise, tags that serve as identifiers for targets are directed to complementary detection probes used with universal detectors of the present invention. Hybridization of an identifier tag to its complementary detection probe on a universal detector generates a signal that indicates the presence of the corresponding target known to be identified by that tag. Accordingly, a sample can be interrogated for the presence of targets of interest using tags and probes of the present invention as follows: a) tags are chosen such that each tag serves as an identifier tag for one target; b) a tag capable of hybridizing to a complementary detection probe will be generated only if the sample being interrogated contains the particular target for which that tag serves as an identifier tag; and c) only a tag generated as a result of the presence of the corresponding target in the sample will hybridize to a detection probe and generate a signal on a universal detector.

One of skill in the art would understand that a set of tags and probes is chosen such that each tag to be used as an identifier tag in a particular application has a complementary detection probe on the universal detector being used in that application. One of skill would also understand that the universal tag assay can be practiced using a set of detection probes that includes detection probes complementary to tags that are not being used in a particular application. For example, in one embodiment a universal detector may advantageously be manufactured with a fixed array of 1000 or more detection probes for use in a wide variety of applications, while a particular application using that universal detector may only use 50-100 identifier tags to interrogate a sample.

It is understood that not only does measuring hybridization of a tag to its complementary detection probe reliably indicate the presence of the corresponding target in a sample, but the absence of hybridization of a tag to its complementary detection probe can also reliably indicate the absence of the corresponding target in a sample. Preferably, at least one internal control is included in a universal tag assay, such that reliably obtaining the expected result from the internal control(s) supports the reliability of results indicating the either the presence or absence of a tag hybridization signal. Multiple internal controls maybe used to increase the reliability and robustness of an assay.

Tag/probe sets may include control sequences that may be used for calibration, quality control, and comparison between experiments. Control sequences may include constant sequences or “housekeeping” sequences that are expected to be present in a sample and generate tagged molecules. If desired, the robustness of the assay may be enhanced by choosing more than one distinct tag to serve as an identifier tag for the same target. In such an embodiment, hybridization of all the identifier tags corresponding to the same target to their complementary detection probes would indicate the presence of the target with a high degree of reliability. Likewise, if none of the identifier tags for the same target hybridize to their complementary tags, then such a signal more reliably indicates the absence of the target in the sample being assayed, especially if other internal controls give positive hybridization signals that indicate suitable reaction conditions. Intermediate results wherein only a few of the identifier tags bind to their complementary detection probes could serve as a signal that the results of the assay are not reliable and reagents or reaction conditions should be examined.

As used herein, the term “tag” generally refers to a molecule capable of binding to a probe, where “tag” may encompass both oligonucleotides in a tagged molecule, tags expressed in computer-readable form, and the concept of tags as disclosed herein. The term identifier tag or “tag sequence” as used herein may describe a string of nucleotides in a molecule, or may describe an information string representing the properties of the string of nucleotides, where such an information string can be manipulated as part of a program for designing or selecting a set of tags having desired properties. Preferably, the information string is in computer-readable form. In the present invention, an “identifier tag” is a tag chosen to serve as a distinct identifier for a particular target. As used herein, the term “identifier tag” is used to refer both to the oligonucleotide that binds to a complementary detection probe and to nucleotide sequence of the identifier tag. The term “complement of an identifier tag” can refer to a string of nucleotides that make up the oligonucleotide having a nucleotide sequence complementary to the nucleotide sequence of the identifier tag, and can also refer to the nucleotide sequence (information string) of the complement.

As used herein, the term “detection probe” generally refers to a molecule capable of binding to a tag, where “detection probe” may encompass probe molecules immobilized to a support, probe molecules not immobilized to a support, probes expressed in computer-readable form, and the concept of detection probes as disclosed herein. More specifically, the term “probe sequence” as used herein refers to the nucleotide sequence of an oligonucleotide probe, and may describe a physical string of nucleotides that make up a sequence, or may describe an information string representing the properties of the string of nucleotides, where such an information string can be manipulated as part of a program for designing or selecting a set of probes having desired properties. Preferably, the information string is in computer-readable form. The term “detection probe” is generally used herein to refer to a tag-complementary probe coupled to a detection means for measuring hybridization of a tag to the detection probe. Preferably, a detection probe is immobilized to a support that includes a detection means. Such a support may include but is not limited to a surface, a film, or a particle, where a surface is preferably a “chip” surface suitable for mounting an array of immobilized probes and having at least one component of the detection means, and a particle is preferably a bead having at least one component of the detection means. “Detection probe” can also refer to a computational model of a tag-complementary probe coupled to a detection means for detecting hybridization. The term “detection probe” may particularly be used herein to distinguish the detection probe from other components also referred to by the term “probe” e.g., RC probes and ligation (LCR) probes.

In accordance with one aspect of the present invention, a set of tag sequences and probe sequences is selected such that a tag having a certain tag sequence will hybridize only to a probe having a sequence that is an exact complement, and no tag will detectably hybridize with any other probe in the set that is not its exact complement. Such a set is referred to herein as a “minimally cross-hybridizing set.” It is understand that due to complementarity, a minimally cross-hybridizing set of tag sequences and probe sequences may be selected on the basis of tag sequence or probe sequence. Preferably, all tag sequences in a set are selected to have the same or substantially the same G-C content, such that all probe/tag duplexes have similar melting temperatures. Preferably, tag sequences are selected such that all probe/tag duplexes have similar stacking energy. Advantageously, such a set will provide tag-probe hybridization reactions with the desired level of selectivity. Even more preferably, such selective hybridization reactions can be carried out under conditions of moderate stringency.

The length of tag (and probe) sequences suitable for a given embodiment can be determined by one of skill in the art. Preferably, the length of tag and probe sequences is determined by the size of the tag/probe set used to interrogate a sample. Generally, the size of the tag/probe set used to interrogate a sample will determine the degree of complexity needed, and tag/probe length is an important determinant of complexity. Generally, the estimated number of targets being tested in a sample will determine the size of the tag/probe set needed for that embodiment. A set of tags and probes suitable for use in the universal chip system may include tags and probes of different lengths, as long as all tags and probes satisfy the hybridization criteria for a given embodiment. For embodiments involving low density arrays wherein about 100 or fewer targets are to be detected, tags having a length of 10, 11, 12, 13, 14, 15, 16, 17, or more than 18 nucleotides may be utilized. Preferably, a tag sequence for a low-density array is 15 nucleotides in length. Tags longer than 18 nucleotides may be used for low density arrays if desired. For embodiments involving higher density arrays wherein hundreds or thousands of target sequences are to be detected, tag and probe sequences may need to be greater than 15 nucleotides in length, in order to provide a sufficiently large set of tags and probes that satisfy the hybridization criteria for a given embodiment.

Algorithms for generating minimally cross-hybridizing sets of tags and probes are known in the art. A set of tags and probes having desired properties may be obtained by following some or all of a series of tag selection steps, as follows: a) determining all possible tag sequences of a selected length, and/or all possible tag sequences with selected hybridization properties b) selecting tag sequences so that all tags differ by at least two nucleotides in the tag sequence string, such that no tag can hybridize to a non-complementary probe with fewer than two mismatches; c) if desired, refining the selection based on the relative destabilizing effects of mismatches at different positions; d) selecting tag sequences so that there is no secondary structure within the complementary probes used to detect the tags; e) selecting tags so that probes complementary to the tags do not hybridize to each other; f) when all tags are the same length, selecting tags so that all tags have substantially the same, and preferably exactly the same, overall base composition (i.e., the same A+T to G+C ratio), so all tag/probe pairs have the same melting temperature; g) when tags are differing lengths, selecting tags having the A+T to G+C ratio that permits all tag/probe pairs to have the same melting temperature. Additional steps not recited here may also be appropriate to obtain a set of tags and probes having desired properties suitable for a particular embodiment.

Selection steps such as those recited above may be performed in various art-recognized ways. Approaches to designing tag/probe sets for use in a particular application include computational “in silico” approaches to model tag and probe behavior, or experimental “in vitro” approaches using biomolecules such as polynucleotides to accomplish tag and probe sorting, or combinations of these approaches.

Computational approaches can be used in which computational algorithms serve as models of biological molecules. Such approaches and algorithms are known in the art. For example, computer programs installed on computers can be used to make the relevant calculations and comparisons, to execute a desired set of selection steps, and to generate a suitable set of sequence tags. Methods for applying a series of selection steps to design a tag/probe set can be found in the art, e.g., as disclosed by Morris et al. (U.S. Pat. No. 6,458,530 and EP 0799897) where a pool of potential tags is generated and a series of pairwise comparisons is carried out to yield a final set of tags that satisfy certain selection criteria. Open-ended computational approaches such as genetic algorithms to generate (locally) optimized populations may be used.

In a preferred embodiment, a universal chip for use in the universal tag assay includes an array of electrically coupled detection probe sequences lacking G (guanosine) bases, thereby permitting electrochemical detection of hybridization of tagged DNA or RNA molecules by detecting G oxidation in tagged molecules (containing G) bound to detection probes, using methods for detecting oxidation-reduction known in the art. For example, G-oxidation in tagged molecules may be detected using transition metal complexes, preferably ruthenium complexes, as disclosed in U.S. Pat. No. 5,871,918. Advantageously, the use of redox-inactive detection probes (e.g., probes lacking G) permits a high density of probes on a universal detector without a background oxidation signal.

Universal Detector

An object of the present invention provides a universal detector having detection probes complementary to identifier tags, where detection probes are coupled to a detection means and the interaction of identifier tags with complementary detection probes indicates the presence or absence of targets in the sample being interrogated. Preferably, a universal detector has an array of detection probes. An “array” is a collection of probes in a known arrangement, and an “array of detection probes” as disclosed herein provides a medium for detecting the presence of targets in a sample based on rules for matching tags and probes, where the rules for matching tags and probes are peculiar to each embodiment. Generally, an array of detection probes refers to an array of probes immobilized to a support, where the sequence (the identity) of each detection probe at each location is known. Alternately, an array of detection probes may refer to a set of detection probes that are not immobilized and can be moved on a surface, or may refer to a set of detection probes coupled to one or more particles such as beads. Preferably, the process of detecting identifier tags hybridized to detection probes is automated. Microarrays having a large of number of immobilized detection probes of known identity can be used for massively parallel gene expression and gene discovery studies. A variety of detection means for measuring hybridization of tags to probes are known in the art, including fluorescent, colorimetric, radiometric, electrical, or electrochemical means.

A further object of the present invention provides a “universal chip” where the term “universal chip” refers generally to a support having arrays of detection probes selected as described above, wherein the detection probes are coupled to a detection means and further wherein hybridization of tags to probes can be detected. In a preferred embodiment, a detection means utilizes electrochemical detection of hybridization of tags to detection probes immobilized to a “universal chip” in a known array. Because the sequence of each detection probe at each location in such an array is known, the sequence of the complementary identifier tag hybridizing to a detection probe is automatically known and thus, is known to indicate the presence of the target corresponding to that tag.

Diverse methods of making oligonucleotide arrays are known, for example as disclosed in U.S. Pat. Nos. 5,412,087, 5,143,854, or 5,384,261 (the entire contents of each of which are hereby expressly incorporated by reference). One object of the present invention provides a universal detector having detection probes attached to a support that functions as an electrical contact surface or electrode to detect hybridization of tags to detection probes. Methods for attaching oligonucleotides to an electrical contact surface are well known, for example as disclosed in any of U.S. Pat. Nos. 5,312,527, 5,776,672, 5,972,692, 6,200,761, or 6,221,586, the entire contents of each of which are hereby expressly incorporated by reference.

In the fabrication process, many other alternative materials and processes can be used. The substrate may be glass or other ceramic material; the bottom silicon dioxide can be replaced by silicon nitride, silicon dioxide deposited by other means, or other polymer materials; the conducting layer can be any appropriate material such as platinum, palladium, rhodium, a carbon composition, an oxide, or a semiconductor. For amperometric measurement either a three-electrode system consisting of a working electrode, a counter electrode and reference electrode or a two-electrode system consisting of a working and a counter/reference electrode may be used to facilitate the measurement. The working electrodes should provide a consistent surface, reproducible response from the redox species of interest, and a low background current over the potential range required for the measurement. The working electrodes may be any suitable conductive materials, preferably noble metals such as gold and platinum, or conductive carbon materials in various forms including graphite, glassy carbon and carbon paste. For a three electrode system the reference electrode is usually silver or silver/silver chloride, and the counter electrode may be prepared from any suitable materials such as noble metals, other metals such as copper and zinc, metal oxides or carbon compositions. Alternatively, the conducting layer can be prepared by screen printing of the electrode materials onto the substrate. Screen printing typically involves preparation of an organic slurry or inorganic slurry of an electrode material, such as a fine powder of carbon or gold, onto the substrate through a fine screen, preferably a silk screen. The electrode material slurry may be fixed on the surface by heating or by air drying. The electrode may be any suitable conductive material such as gold, carbon, platinum, palladium, indium-tin-oxide. It is often advantageous to coat the electrode surface with a material such as avidin, streptavidin, neutravidin, or other polymers, to increase the immobilization of detection probes. Methods for the attachment include passive adsorption and covalent attachment.

If gold is chosen for the conducting layer, the layer can be evaporated, sputtered, or electroplated. A low temperature oxide layer can be replaced by spin-on dielectric materials or other polymer materials such as polyimide, or parylene. Reagent and electrical connections can be on the same side of a chip or on adjacent sides, though the opposite side configuration is preferred. Materials, temperatures, times, and dimensions may be altered to produce detectors, preferably chips, having substantially the same properties and functionality, as will be appreciated by those of skill in the art. Materials, temperatures, times, and dimensions may be altered by one of skill in the art to produce chips having the properties desired for any particular embodiment.

In a preferred embodiment, the detection probes are immobilized on a support having an array of electrodes sandwiched between two layers of silicon dioxide insulator attached to the silicon substrate, where a supporting layer is opposite the silicon substrate and the chip is oriented such that the silicon substrate is on the top and the supporting layer is on the bottom, as disclosed in U.S. patent application Ser. No. 10/121,240 the entire contents of which are hereby incorporated by reference. Preferably, gold electrodes are used. Alternately, carbon electrodes such as graphite, glassy carbon, and carbon paste can be used. In this preferred embodiment, access to the surfaces of the working electrodes, where the detection probes are immobilized, is through windows through the silicon substrate and top layer of insulator on the top surface of the chip. Windows on the underside (etched through the supporting layer and the bottom layer of insulator) allow access to a counter (or detector) electrode and a reference electrode. For gold electrodes, the two types of electrodes in the chip are selectively interconnected by deposited gold wiring within the insulating layer or by other methods known in the art. Access to the working electrode, reference electrode, and counter electrode allows a complete circuit to be formed which will enable standard techniques in the art, such as amperometric measurements, to be performed using the chip. An electrode potential applied to the working electrode, where the electrochemically active materials are present through association with the detection probes and tag sequences, will produce current proportional to the amount of tag sequence attached to the detection probes.

Electrochemical Readout: Measuring Hybridization of Identifier Tags to Complementary Detection Probes

Another aspect of the invention provides detection means for measuring hybridization of tags to detection probes. In one embodiment, DNA hybridization is detected by an electrochemical method, which generally includes observing the redox behavior of a single-stranded DNA detection probe as compared to a double-stranded DNA. For example, a voltammetric sequence-selective sensor can be used for detecting a target nucleic acid, where a double-stranded nucleic acid is contacted to a redox-active complex for example as disclosed in U.S. Pat. No. 5,312,527, the entire contents of which are hereby incorporated by reference. The complex binds non-specifically to the double-stranded DNA, and because the complex itself is the redox-active compound that provides a voltammetric signal, the complex does not function in a catalytic manner without the addition of an enzyme. Alternately, an electrochemical assay for nucleic acids can be used, in which a competitive binding event between a ligand and an antiligand is detected electrochemically, as disclosed in U.S. Pat. No. 4,840,893, the entire contents of which are hereby incorporated by reference.

In another embodiment, RNA hybridization is detected by an electrochemical method, which generally includes observing the redox behavior of a single-stranded DNA detection probe as compared to a DNA/RNA duplex formed by hybridization of an RNA tag to a DNA detection probe.

Hybridization of tags and probes may be detected using a transition metal complex capable of oxidizing at least one oxidizable base in an oxidation-reduction reaction under conditions that cause an oxidation-reduction reaction between the transition metal complex and the oxidizable base, where the probe or the tagged molecule or both contain at least one oxidizable base. The oxidation-reduction reaction indicating hybridization is detected by measuring electron transfer from each oxidized base, as disclosed in U.S. Pat. No. 5,871,981, the entire contents of which are hereby incorporated by reference.

In a preferred embodiment, hybridization of identifier tags to DNA detection probes immobilized on gold or other electrodes may be carried out using methods disclosed by Steele et al. (1998, Anal. Chem 70:4670-4677). Preferably, multivalent ions with 2, 3, or 4 positive charges are used, which are capable of electrochemical detection by direct reaction without affecting the nucleic acid. In the preferred embodiment these ions bind electrostatically to nucleic acid phosphate irrespective of whether it is in the double-helical or single-stranded form. The presence or absence of hybridized identifier tag DNA is determined for each detection probe, based on electron transfer measurements taken at each detection probe site. The sample being interrogated may be contacted with the oligonucleotide detection probe in any suitable manner known to those skilled in the art. By way of example, a DNA sample being interrogated for the presence of target nucleotide sequences may be in solution and the oligonucleotide detection probes immobilized on a solid support, whereby the DNA sample may be contacted with the oligonucleotide detection probe by immersing the solid support having the oligonucleotide detection probes immobilized thereon in the solution containing the DNA sample. Suitable transition metal complexes that bind nucleic acid electrostatically and whose reduction or oxidation is electrochemically detectable in an appropriate voltage regime include Ru(NH₃)₆³⁺, Ru(NH₃)₅ pyridine³⁺ and other transition metal complexes that can be determined by one of skill in the art.

In accordance with another aspect of the present invention, oligonucleotide detection probe sequences may be designed to be redox inactive, or to have very low redox activity, for example as disclosed in U.S. Pat. No. 5,871,918. In one embodiment, oligonucleotide probe sequences are designed so as to not contain G (guanosine) bases, permitting electrochemical detection of hybridization of tagged DNA molecules by detecting G oxidation in tagged molecules with identifier tags hybridized to their probe complements, as disclosed in U.S. Pat. No. 5,871,918. Advantageously, the use of redox-inactive probes permits a high density of probes on a universal detector without a background oxidation signal. In some embodiments, the detection probes may include one or more uncharged nucleotide analogs such as peptide nucleic acids (PNAs), phosphotriesters, methylphosphonates. These nucleic acid analogs are known in the art.

In particular, PNAs are discussed in: Nielsen, “DNA analogues with nonphosphodiester backbones,” Annu Rev Biophys Biomol Struct, 1995; 24:167-83; Nielsen et al., “An introduction to peptide nucleic acid,” Curr Issues Mol Biol, 1999; 1(1-2):89-104; Ray et al., “Peptide nucleic acid (PNA): its medical and biotechnical applications and promise for the future,” FASEB J., 2000 June; 14(9):1041-60; all of which are hereby expressly incorporated by reference in their entirety.

Phophotriesters are discussed in: Sung et al., “Synthesis of the human insulin gene. Part II. Further improvements in the modified phosphotriester method and the synthesis of seventeen deoxyribooligonucleotide fragments constituting human insulin chains B and mini-CDNA,” Nucleic Acids Res, Dec. 20, 1979; 7(8):2199-212; van Boom et al., “Synthesis of oligonucleotides with sequences identical with or analogous to the 3′-end of 16S ribosomal RNA of Escherichia coli: preparation of m-6-2-A-C-C-U-C-C and A-C-C-U-C-m4-2C via phosphotriester intermediates,” Nucleic Acids Res, 1977 March; 4(3):747-59; Marcus-Sekura et al., “Comparative inhibition of chloramphenicol acetyltransferase gene expression by antisense oligonucleotide analogues having alkyl phosphotriester, methylphosphonate and phosphorothioate linkages,” Nucleic Acids Res, Jul. 24, 1987; 15(14):5749-63; all of which are hereby expressly incorporated by reference in their entirety.

Methylphosphonates are discussed in: U.S. Pat. No. 4,469,863 (Ts'o et al.); Lin et al., “Use of EDTA derivatization to characterize interactions between oligodeoxyribonucleoside methylphophonates and nucleic acids,” Biochemistry, Feb. 7, 1989; 28(3):1054-61; Vyazovkina et al., “Synthesis of specific diastereomers of a DNA methylphosphonate heptamer, d(CpCpApApApCpA), and stability of base pairing with the normal DNA octamer d(TPGPTPTPTPGPGPC),” Nucleic Acids Res, Jun. 25, 1994; 22(12):2404-9; Le Bec et al., “Stereospecific Grignard-Activated Solid Phase Synthesis of DNA Methylphosphonate Dimers,” J Org Chem, Jan 26, 1996; 61(2):510-513; Vyazovkina et al., “Synthesis of specific diastereomers of a DNA methylphosphonate heptamer, d(CpCpApApApCpA), and stability of base pairing with the normal DNA octamer d(TPGPTPTPTPGPGPC),” Nucleic Acids Res, Jun. 25, 1994; 22(12):2404-9; Kibler-Herzog et al., “Duplex stabilities of phosphorothioate, methylphosphonate, and RNA analogs of two DNA 14-mers,” Nucleic Acids Res, Jun. 11, 1991; 19(11):2979-86; Disney et al., “Targeting a Pneumocystis carinii group I intron with methylphosphonate oligonucleotides: backbone charge is not required for binding or reactivity,” Biochemistry, Jun. 13, 2000; 39(23):6991-7000; Ferguson et al., “Application of free-energy decomposition to determine the relative stability of R and S oligodeoxyribonucleotide methylphosphonates,” Antisense Res Dev, 1991 Fall; 1(3):243-54; Thiviyanathan et al., “Structure of hybrid backbone methylphosphonate DNA heteroduplexes: effect of R and S stereochemistry,” Biochemistry, Jan. 22, 2002; 41(3):827-38; Reynolds et al., “Synthesis and thermodynamics of oligonucleotides containing chirally pure R(P) methylphosphonate linkages,” Nucleic Acids Res, Nov. 15, 1996; 24(22):4584-91; Hardwidge et al., “Charge neutralization and DNA bending by the Escherichia coli catabolite activator protein,” Nucleic Acids Res, May 1, 2002; 30(9):1879-85; Okonogi et al., “Phosphate backbone neutralization increases duplex DNA flexibility: A model for protein binding,” PNAS U.S.A., Apr. 2, 2002; 99(7):4156-60; all of which are hereby incorporated by reference.

The occurrence of the oxidation-reduction reaction may be detected according to any suitable means known to those skilled in the art. For example, the oxidation-reduction reaction may be detected using a detection electrode to observe a change in the electronic signal which is indicative of the occurrence of the oxidation-reduction reaction. Suitable reference electrodes will also be known in the art and include, for example, silver, silver/silver chloride electrodes. The electronic signal associated with the oxidation-reduction reaction permits the determination of the presence or absence of hybridized tags by measuring the Faradaic current or total charge associated with the occurrence of the oxidation-reduction reaction. The current depends on the presence of the positively charged redox ion closely associated with the electrode, which in turn depends on the amount of nucleic acid phosphate hybridized to the electrode. The electronic signal may be characteristic of any electrochemical method, including cyclic voltammetry, normal pulse voltammetry, differential pulse voltammetry, chronoamperometry, and square-wave voltammetry. The amount of hybridized DNA is determined by subtracting the current or total charge characteristic of the probes and other molecules bound to the electrode in the starting state from the current or total charge measured after the hybridization reaction.

Enhancing Detection with Development Reagents

As noted above, one or more development reagents can be included to enhance or otherwise optimize electrochemical detection of signals in a universal tag assay.

### EXAMPLES

### Example 1

### SNP Genotyping Using Serial ICR and RC Amplification

In order to determine the genotype of a diploid organism, a genomic DNA sample is assayed to determine which polymorphic nucleotides are present at a particular SNP site. As shown in FIG. 1, the SNP may have T or G at the polymorphic position. The assay has four steps: 1) carrying out a first ICR directly on genomic DNA containing the target SNP site; 2) using a probe 5′ flap released from the first ICR in a second ICR using a hairpin cassette, and removing uncleaved generic-sequence hairpin cassettes via capture of a 3′-end moiety, e.g., capture of 3′-biotin-labelled hairpin cassettes onto a streptavidin (SA) surface; 3) RC amplification using 5′ flaps released from second ICR as polymerization primers and two pre-formed circular oligonucleotides as RC probes, where each probe contains the complement of the identifier tag sequence that corresponds to a particular allele; and 4) hybridization of identifier tags present in RC amplification products to complementary detection probes on a universal detector, and electrochemical detection of hybridization. The reaction can be carried out isothermally at about 65° C.

The first ICR in Step 1 (FIG. 1) utilizes a set of oligonucleotides, namely a probe and an upstream oligonucleotide, that bind to target nucleotide sequence flanking and including the SNP, where the probe and the upstream oligonucleotide overlap at the polymorphic site to form an bifurcated complex on the template. A structure-specific 5′ nuclease recognizes and cleaves the bifurcated cleavage structure, releasing a 5′ flap that is not complementary to the target nucleotide sequence.

In Step 2, which can be run simultaneously with step 1, the 5′ flaps from the first ICR serve as upstream oligonucleotides in a second ICR using hairpin cassettes specific for each allele. Formation of a bifurcated cleavage structure on a hairpin cassette results in cleavage and release of a 5′-flap from the hairpin cassette. Uncleaved hairpin cassettes can be removed to preclude competition for the polymerization primer binding site on the circular RC probes used in Step 3. Here, each hairpin cassette is 3′-labelled with biotin, and the cassettes, both uncleaved and the cleaved fragments containing the hairpin, are removed using SA-coated beads or plate. In another embodiment, a molar excess of pre-formed circular RC probes over hairpin cassettes could be added to enable both RC amplification via the cleaved 5′ flaps and binding of uncleaved hairpin cassettes. In, this format, a 3′-end moiety on the hairpin cassette could prevent background RC amplification when it is bound to the pre-formed circular RC probe.

In Step 3, the cleaved 5′-flaps from the second ICR in Step 2 bind to preformed circular RC probes and serve as polymerization primers for RC amplification, resulting in amplification of the complement to the RC probe sequence. The RC probes contain the complement of at least one distinct identifier tag chosen to correspond to a particular target. In this embodiment, P1 has been chosen as the identifier tag for the T allele, and P2 has been chosen as the identifier tag for the G allele. RC probes can also contain restriction enzyme recognition sites for trimming RC amplification products to generate smaller tagged molecules containing identifier tags.

In Step 4, hybridization of identifier tags to complementary detection probes attached to a universal detector is measured by ruthenium amperometry. As shown in FIG. 1, a hybridization signal from detection probe C1 reports that the P1 identifier tag was generated and bound to C1, which indicates that the T allele is present in the sample. A hybridization signal from detection probe C2 reports that the P2 identifier tag was generated and bound to C2, which indicates that the G allele is present in the sample. Hybridization signals from both C1 and C2 indicate that both the T allele and the G allele are present in the sample, i.e., the organism from which the genomic DNA was obtained is heterozygous at this SNP site. When a signal is only detected from C1, then the organism is homozygous for the T allele, and when a signal is only detected from C2, then the organism is homozygous for the G allele.

### Example 2

### SNP Genotyping Using ICR, Ligation, and RC Amplification

In order to determine the genotype of a diploid organism, a genomic DNA sample is assayed to determine which polymorphic nucleotides are present at a particular SNP site. The 5′ flap, generated in an allele-specific manner in an ICR (FIG. 2, Step 1) or serial ICR, can be hybridized to a circularizing template (RC probe/flap template) that contains sequence complementary to the 5′ flap (FIG. 2, Step 2). Other regions of the circularizing template contain generic sequence that enables hybridization of an uncircularized RC probe, which contains a 5′-phosphate. The 5′ end of the ICR downstream probe would also contain a 5′ phosphate so that the cleaved 5′ flap can be doubly ligated to the uncircularized RC probe, thus yielding a circularized RC probe. An RC amplification reaction (FIG. 2, Step 3) using either an RNA polymerase to generate single-stranded RNA from a promoter on the RC probe/flap template or a promoter generated by extending the RC probe/flap template or using an RCA primer (RC probe/flap template itself or another primer that hybridizes to the circularized probe) and DNA polymerase to generate single-stranded DNA can be performed on the circularized RC probe. In embodiments employing a promoter, the promoter may be a T7 promoter, an SP6 promoter, a T3 promoter or a promoter from which any desired RNA polymerase can initiate transcription. The complement (C1 or C2) of the identifier tag sequence is present in another region of the uncircularized/circularized RC probe (shown in FIG. 2, Step 2). In some embodiments, the RC probe/flap template may be the detection probe on the universal chip, and the 3′ terminus of the detection probe may be extended to detect the presence of the target in the sample.

In another embodiment, an integrated ICR probe comprising both the upstream and downstream ICR probes forms an overlapped substrate with the SNP site in the target (FIG. 3, Step 1), wherein the substrate also contains a 5′ flap. Allele-specific cleavage of the 5′ flap and subsequent allele-specific ligation (FIG. 3, Step 2) yields a circularized RC probe. The integrated ICR probe, which is converted to a circularized RC probe in an allele-specific manner, can also contain a complementary sequence (C1 or C2) to the identifier tag sequence (P1 or P2). Performing RC amplification on the circularized product (FIG. 3, Step 3) of the 5′-nuclease/ligase reactions would then yield either single-stranded RNA (using an RNA polymerase) or single-stranded DNA (using a DNA polymerase and RC amplification primer) containing multiple copies of the identifier tag (P1 or P2).

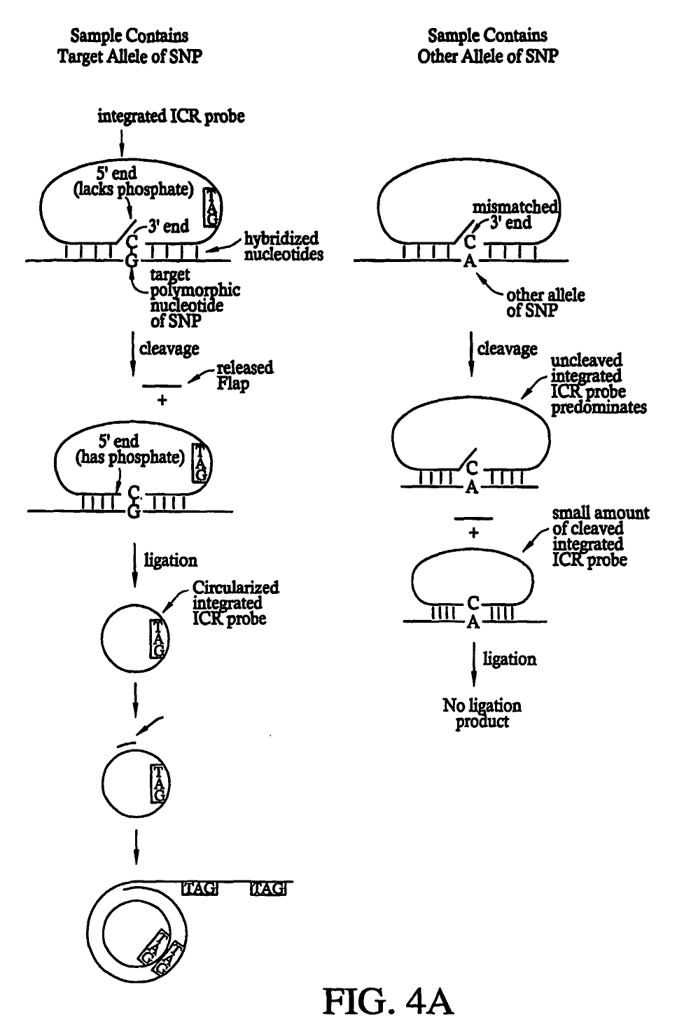

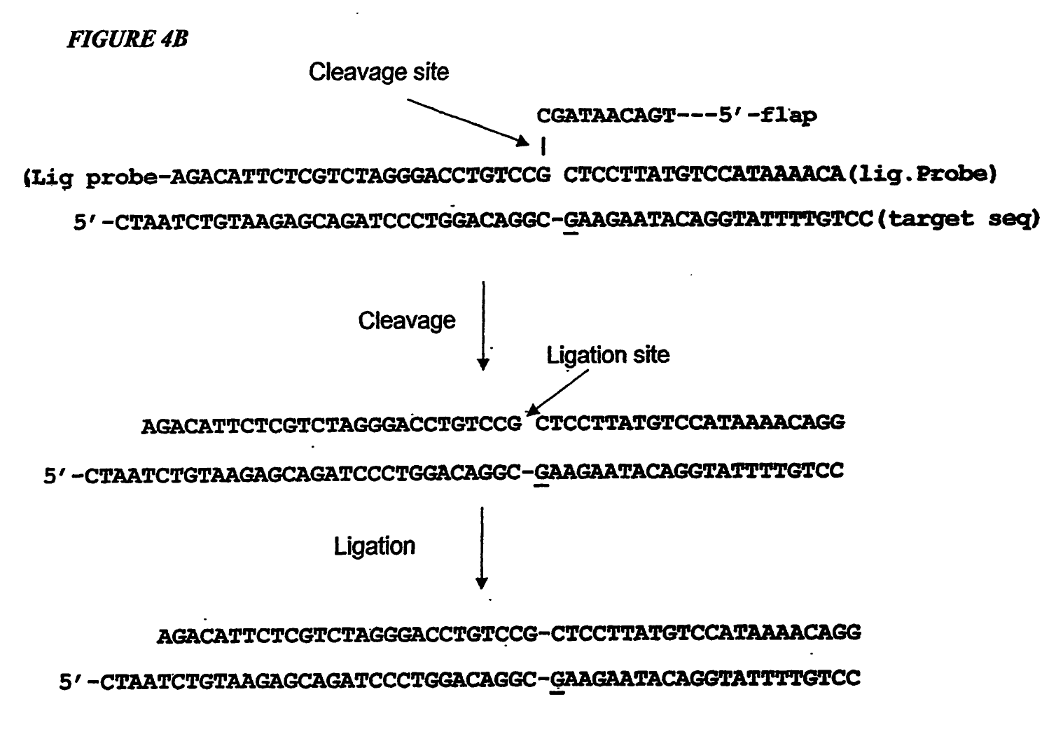

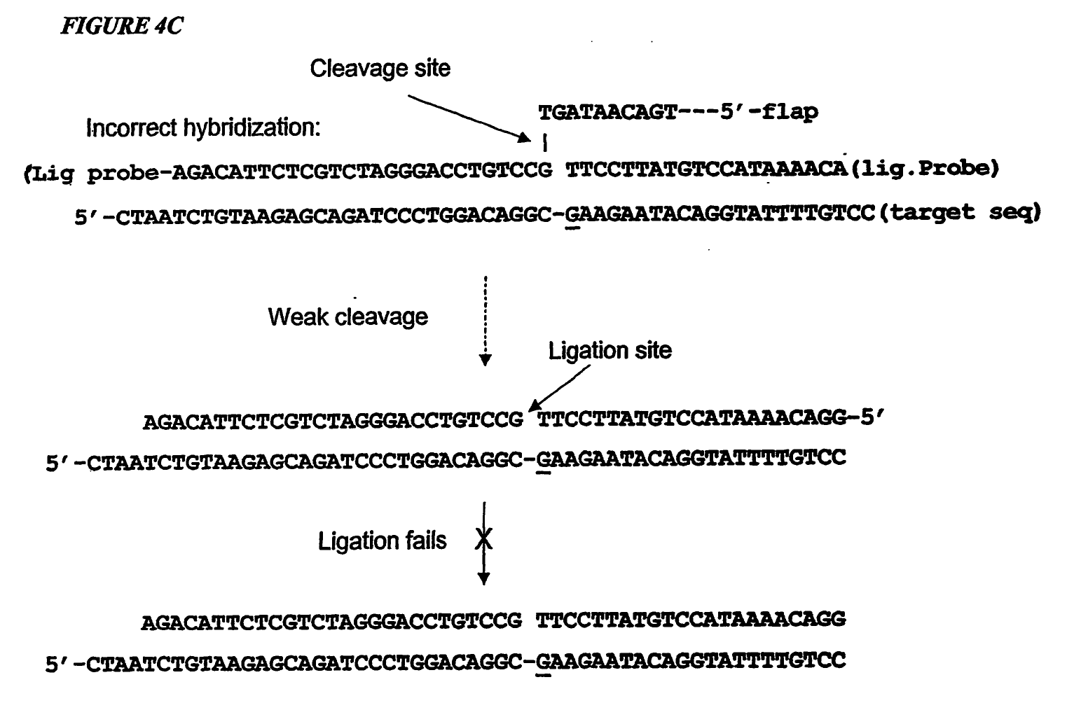

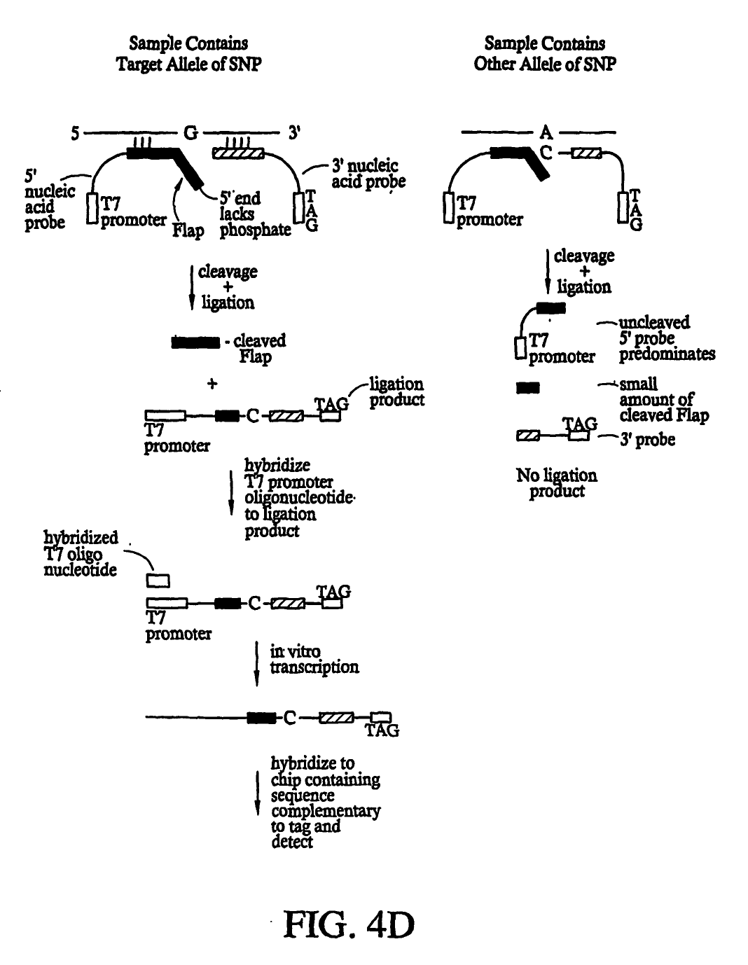

In another embodiment utilizing a circularizable integrated ICR probe, the 5′ region of the integrated ICR probe comprises a first sequence which is complementary to a first portion of the target nucleotide sequence and a second sequence 5′ of the first sequence which is not complementary to the target nucleotide sequence. The 3′ end of the circularizable integrated ICR probe comprises a third sequence which is complementary to a second portion of the target nucleotide sequence. In some embodiments, the 5′ end of the circularizable integrated ICR probe lacks a phosphate. In some embodiments, the second portion of the target nucleotide sequence is immediately adjacent to the first portion of the target nucleotide sequence, but, in other embodiments the first portion of the target nucleotide sequence may not be immediately adjacent to the second portion of the target nucleotide sequence. In some embodiments, the target nucleotide sequence comprises a polymorphic nucleotide and either the first sequence of the 5′ region of the integrated ICR probe or the third sequence of the 3′ end of the integrated ICR probe contains a nucleotide which is complementary to one allele of the polymorphic nucleotide but not complementary to other alleles of the polymorphic nucleotide such that the first sequence or the third sequence will form a perfect hybrid with one allele but will have a mismatch when hybridized to the other alleles. Preferably, the nucleotide complementary to one allele of the polymorphic nucleotide is within about 5 nucleotides on either side of the ligation site. In one embodiment, the third sequence in the integrated ICR probe comprises the nucleotide complementary to the polymorphic nucleotide at its 3′ end. In some embodiments, the integrated ICR probe also comprises a nucleotide sequence complementary to an identifier tag. One method for utilizing this embodiment of the integrated ICR probe is illustrated in FIGS. 4A-4C.

As illustrated in FIGS. 4A and 4B, the first sequence in the 5′ region of the integrated ICR probe hybridizes to the target nucleotide sequence while the second sequence in the 5′ region forms a 5′ flap since it is not complementary to the target nucleotide sequence. In some embodiments, the 5′ terminus lacks a phosphate and is thus incapable of being ligated to the hydroxyl group at the 3′ end. The third sequence in the 3′ region of the integrated ICR probe also hybridizes to the target nucleotide sequence. In the embodiment illustrated in FIGS. 4A-4C, the third sequence in the 3′ region of the integrated ICR probe contains the polymorphic base of a SNP at its 3′ end. Thus, the third sequence is perfectly complementary to one allele of the SNP but will have a mismatch at its 3′ end when hybridized to the other allele of the SNP. If the sample contains the allele of the SNP which is perfectly complementary to the 3′ region of the integrated ICR probe, the 5′ flap will be efficiently cleaved, generating a 5′ phosphate which can be ligated to the 3′ hydroxyl, thereby generating a circular molecule (See FIGS. 4A and 4B). A primer is hybridized to the circularized molecule and rolling circle amplification is performed to generate an amplification product. In embodiments in which the integrated ICR probe contains a sequence complementary to an identifier tag, the amplification product contains the identifier tag. The amplification product is detected using any of the methods described herein to indicate the presence of the target allele of the SNP in the sample. As an alternative to performing a rolling circle amplification reaction on the circularized ICR probe, in some embodiments, the 5′ flap released after cleavage is used to generate an amplification product using any of the methods described herein. For example, in some embodiments, the cleaved flap is used as a primer in a rolling circle amplification procedure as described herein. In other embodiments, the cleaved flap comprises a promoter or a sequence complementary to a promoter from which transcription is initiated to indicate the presence of the target nucleotide sequence in the sample as described herein.

In contrast, if the sample contains the allele of the SNP which is not perfectly complementary to the 3′ region of the integrated ICR probe, the nucleotide at the 3′ end of the ICR probe will be mismatched (See FIGS. 4A and 4C). Because of the mismatched nucleotide at the 3′ end, cleavage of the 5′ flap will be inefficient. In addition, even if the 5′ flap is cleaved, the mismatch at the 3′ end of the integrated ICR probe will prevent ligation from occurring. In the absence of circular ligation products, no amplification product will be generated.

In some embodiments, rather than using an integrated ICR probe, the method is performed using separate nucleic acid molecules. In this embodiment, a 5′ nucleic acid probe comprising a first sequence which is complementary to a first portion of the target nucleotide sequence and a second sequence 5′ of the first sequence which is not complementary to the target nucleotide sequence is hybridized to the nucleic acids in the sample. A 3′ nucleic acid probe which is a separate molecule from the 5′ nucleic acid probe and which comprises a third sequence which is complementary to a second portion of the target nucleotide sequence is also hybridized to the nucleic acid in the sample. In some embodiments, the 5′ end of the 5′ nucleic acid probe lacks a phosphate. In some embodiments, the 5′ nucleic acid probe comprises one strand of a promoter positioned 3′ of the first sequence. In some embodiments, the second portion of the target nucleotide sequence is immediately adjacent to the first portion of the target nucleotide sequence, but, in other embodiments the first portion of the target nucleotide sequence may not be immediately adjacent to the second portion of the target nucleotide sequence. In some embodiments, the target nucleotide sequence comprises a polymorphic nucleotide and either the first sequence of the 5′ nucleic acid probe or the third sequence of the 3′ nucleic acid probe contains a nucleotide which is complementary to one allele of the polymorphic nucleotide but not complementary to other alleles of the polymorphic nucleotide such that the first sequence or the third sequence will form a perfect hybrid with one allele but will have a mismatch when hybridized to the other alleles. Preferably, the nucleotide complementary to one allele of the polymorphic nucleotide is within about 5 nucleotides on either side of the ligation site. In one embodiment, the third sequence in the 3′ nucleic acid probe comprises the nucleotide complementary to the polymorphic nucleotide at its 3′ end. In some embodiments, the 3′ nucleic acid probe also comprises a nucleotide sequence complementary to an identifier tag. One method for utilizing this embodiment is illustrated in FIG. 4D.

As illustrated in FIG. 4D, the first sequence in the 5′ nucleic acid probe hybridizes to the target nucleotide sequence while the second sequence in the 5′ nucleic acid probe forms a 5′ flap since it is not complementary to the target nucleotide sequence. In some embodiments, the 5′ terminus of the 5′ nucleic acid probe lacks a phosphate and is thus incapable of being ligated to the hydroxyl group at the 3′ end of the 3′ nucleic acid probe. The third sequence in the 3′ nucleic acid probe also hybridizes to the target nucleotide sequence. In the embodiment illustrated in FIG. 4D, the third sequence in the 3′ nucleic acid probe contains the polymorphic base of a SNP at its 3′ end. Thus, the third sequence is perfectly complementary to one allele of the SNP but will have a mismatch at its 3′ end when hybridized to the other allele of the SNP. If the sample contains the allele of the SNP which is perfectly complementary to the 3′ nucleic acid probe, the 5′ flap of the 5′ nucleic acid probe will be efficiently cleaved, generating a 5′ phosphate which can be ligated to the 3′ hydroxyl of the 3′ nucleic acid probe, thereby generating a ligation product (See FIG. 4D). In the embodiment illustrated in FIG. 4D, the 5′ probe comprises one strand of a T7 promoter and the 3′ probe comprises a nucleic acid complementary to a tag. The ligation product is then hybridized to a nucleic acid comprising the other strand of the T7 promoter and transcription is initiated to generate a transcription product comprising the tag. The transcription product is detected using any of the methods described herein to indicate the presence of the target allele of the SNP in the sample. As an alternative to performing a transcription reaction, in some embodiments, the 5′ flap released after cleavage is used to generate an amplification product using any of the methods described herein. For example, in some embodiments, the cleaved flap is used as a primer in a rolling circle amplification procedure as described herein. Other embodiments of first and second nucleic acid probes and methods for generating and detecting ligation products comprising them are described in U.S. Provisional Patent Application Ser. No. 60/497,821, entitled Oligonucleotide Sequestering Agents and Methods of Use, filed Aug. 25, 2003, the disclosure of which is incorporated herein by reference in its entirety.

In contrast, if the sample contains the allele of the SNP which is not perfectly complementary to the third sequence in the 3′ nucleic acid probe, the nucleotide at the 3′ end of the 3′ nucleic acid probe will be mismatched (See FIGS. 4D). Because of the mismatched nucleotide at the 3′ end, cleavage of the 5′ flap will be inefficient. In addition, even if the 5′ flap is cleaved, the mismatch at the 3′ end of the 3′ nucleic acid probe will prevent ligation from occurring. In the absence of ligation products, no transcription product comprising the tag will be generated.

The foregoing procedures provide a high degree of sensitivity and specificity. Specificity is provided by a dual filter against the generation of false positives. First because of the mismatch at the 3′ end of the integrated ICR probe, cleavage of the 5′ flap is inefficient. In addition, even if cleavage occurs, the mismatched 3′ end will not ligate to the 5′ phosphate, thereby providing a second level of protection against false positives. The high degree of specificity allows the detection of alleles present at low frequency in the sample, such as mutant alleles which give rise to cancer. In some embodiments, the foregoing procedure is used to detect a mutation present at a level of about 1 part in 10² (i.e. there is one mutant version of the gene in the sample per 100 total versions of the gene in the sample) 1 mutation in the target gene per 100 copies of the gene) or less, about 1 part in 10⁴ or less, about 1 part in 10⁵ or less, or about 1 part in 10⁶ or less.

### Example 3

### SNP Genotyping Using RC Amplification Product as Template for ICR

An RC amplification step is performed on genomic DNA in linear or exponential mode to generate multimeric copies of the RC probe, including repeating copies of target nucleotide sequence (FIG. 5, Step 1). If exponential RC amplification is used, the double stranded product is denatured to generate single stranded product. ICR is then carried out using RC amplification product as a template (FIG. 5, Step 2A or 2B). Target-specific upstream oligonucleotides and downstream probes hybridize to target sequences on the multimeric RCA product. The ICR upstream oligonucleotide and probe can be designed to bind one of two generic sequence/target-specific sequence junctions while still reporting out the genotype of the SNP site. Probe 5′ flaps containing identifier tags are released upon cleavage, where the 5′ flaps function as tagged molecules in a universal tag assay (FIG. 5, Step 3). Uncleaved probes containing the identifier tags can be captured onto a streptavidin surface via a 3′-biotin moiety prior to adding the amplification reaction to the chip. Cleaved 5′ flaps containing identifier tags hybridize to complementary detection probes on a universal detector. Hybridization is electrochemically detected by ruthenium amperometry.

In a variation that would further enhance amplification of 5′ flaps containing identifier tags, serial ICR can be carried out using 5′-flaps released from a first target-dependent ICR carried out on the RC amplification product template. 5′ flaps released from the second ICR function as tagged molecules in a universal tag assay, as described above.

### Example 4

### SNP Genotyping Using Serial ICR and Development Reagent

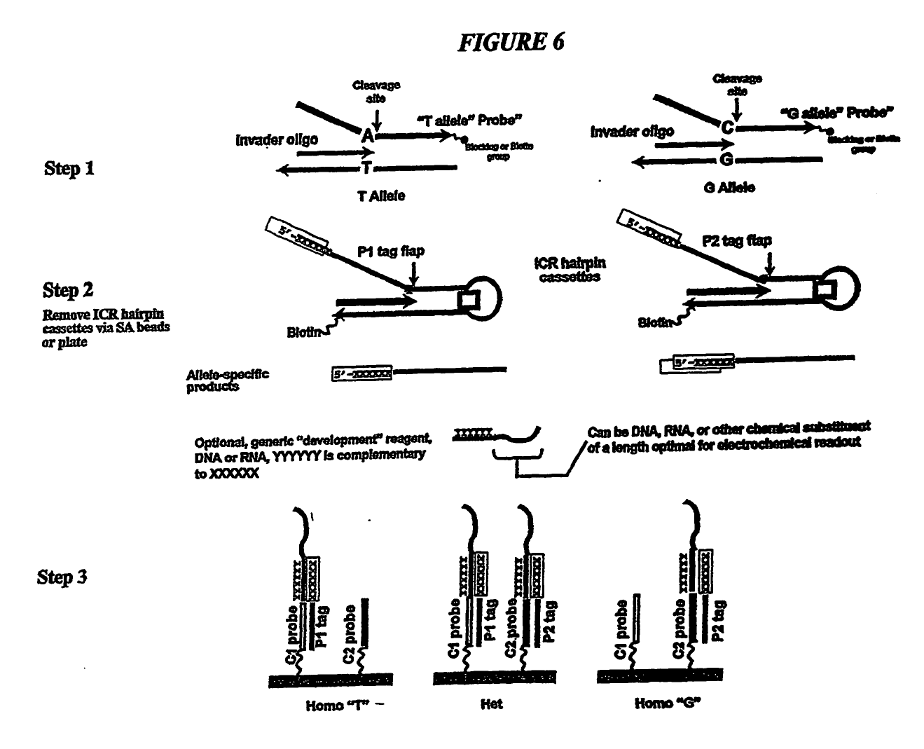

A DNA sample is assayed to determine which polymorphic nucleotides at a particular SNP site are present. As shown in FIG. 6, the SNP may have T or G at the polymorphic position. Template may be genomic DNA from the organism being genotyped, or may be an amplification product, e.g., a PCR product, containing target nucleotide sequence. In Step 1, target-dependent ICR is carried out, wherein upstream oligonucleotides and probes hybridize to template and cleavage by a 5′-nuclease releases the 5′ flap of the probe. In Step 2, a second ICR is carried out using biotinylated hairpin cassettes, where the 5′ flap released from the first ICR functions as the upstream oligonucleotide. After cleavage, hairpin cassettes are removed using a streptavidin-coated surface, e.g., beads. Each 5′ flap released from the hairpin cassette contains an identifier tag, which could function as a tagged molecule in a universal tag assay, and an additional sequence that could be used for further signal enhancement with adevelopment reagent.

Here, each complementary detection probe includes sequence complementary to the identifier tag and sequence complementary to the development reagent. Tagged molecules are incubated with a universal detector in the presence of a ruthenium complex such as Ru(NH₃)⁶⁺, and hybridization is detected electrochemically. As shown in FIG. 6, a hybridization signal from detection probe C1 reports that the P1 identifier tag was generated and bound to C1, which indicates that the T allele is present in the sample. A hybridization signal from detection probe C2 reports that the P2 identifier tag was generated and bound to C2, which indicates that the G allele is present in the sample. Hybridization signals from both C1 and C2 indicate that both the T allele and the G allele are present in the sample, i.e., the organism from which the genomic DNA was obtained is heterozygous at this SNP. When a signal is only detected from C1, then the organism is homozygous for the T allele, and when a signal is only detected from C2, then the organism is homozygous for the G allele.

Further amplification of the electrochemical signal may be obtained by adding an oligonucleotide that contains a generic sequence, which would bind to the overhanging 5′ end of the 5′ flap and containing the complementary sequence. In another embodiment, the interaction of the 5′ region of the 5′ flap and an added development reagent could be via an antigen-antibody interaction. Tagged molecules containing a chemical moiety that binds the development reagent are incubated with a universal detector in the presence of a ruthenium complex such as Ru(NH₃)⁶⁺, and hybridization is detected electrochemically.

### Example 5

### Target Detection Using ICR and Development Regent

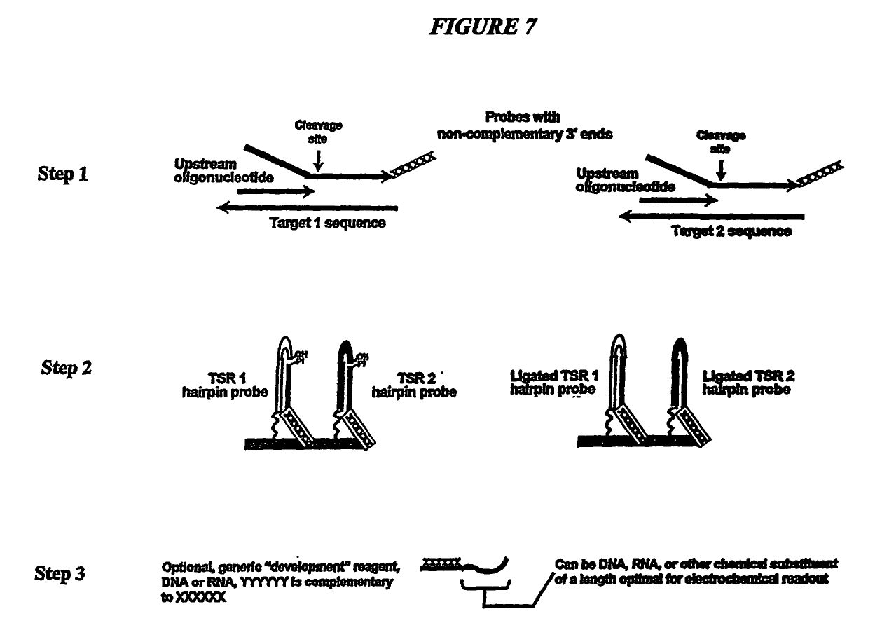

In another embodiment using a development reagent (FIG. 7), a ICR or serial ICR can be performed on the genomic DNA or DNA amplification product (e.g., PCR product or exponential RC amplification product). In this format targets of different sequence are detected using the cleaved 3′ end fragment of the ICR downstream probe. The cleaved 3′ end fragment will contain a 5′ phosphate group, which can hybridize and be ligated to target-specific hairpin detection probes on the detector surface (FIG. 7, Step 2). Stringent washing after hybridization and ligation of the ICR-specific cleavage products could remove background signal from uncleaved ICR downstream probes. A development reagent, described previously, could be used in this format by inclusion of a non-complementary 3′ region in the ICR downstream probe or alternatively 3′ end moiety that can interact with the development reagent.

### Example 6

### SNP Genotyping Using ICR Followed by Ligation and Transcription

In order to determine the genotype of a diploid organism, a genomic DNA sample is assayed to determine which polymorphic nucleotides are present at a particular SNP site. As illustrated in FIG. 8, a target SNP may have T or G at the polymorphic position. In this assay, serial ICR is carried out as shown in FIG. 8, Step 1, using methods as previously described. Template is genomic DNA or DNA amplification product (e.g., PCR product or exponential RC amplification product). The 5′ flaps released from the second ICR, containing either C1 or C2 detection probe sequences, are used to create templates for transcription by RNA polymerase. 5′ flaps released from the ICR hairpin cassettes hybridize to complementary sequence in biotinylated hairpin transcription cassettes that contain a double stranded RNA polymerase promoter site, and the hybridized 5′ flaps are then ligated to the hairpin cassette via the 5′ flap's 3′ hydroxyl group and the biotinylated hairpin transcription cassette's 5′ phosphate group (FIG. 8, Step 2). After stringent washing to remove uncleaved/unligated ICR hairpin cassettes, which could not be ligated but could generate background, RNA polymerase (e.g., T7 polymerase) is added to generate RNA from hairpin transcription cassettes with ligated tag sequence (Step 3). These RNA molecules, containing allele-specific identifier tags, are tagged RNA molecules suitable for use in a universal tag assay. Alternate versions that can be contemplated by one skilled in the art are use of other RNA polymerases and their respective promoter sequences (e.g., SP6 and T3) and the use of generic sequences in the transcription cassette for utilization of development reagent at the chip detection step.

Hybridization of tagged RNA molecules to complementary detection probes is detected by electrochemical readout (FIG. 8, Step 4), e.g., by incubation with a universal detector in the presence of a ruthenium complex. As shown in FIG. 6, a hybridization signal from detection probe C1 reports that the P1 identifier tag was generated and bound to C1, which indicates that the T allele is present in the sample. A hybridization signal from detection probe C2 reports that the P2 identifier tag was generated and bound to C2, which indicates that the G allele is present in the sample. Hybridization signals from both C1 and C2 indicate that both the T allele and the G allele are present in the sample, i.e., the organism from which the genomic DNA was obtained is heterozygous at this SNP. When a signal is only detected from C1, then the organism is homozygous for the T allele, and when a signal is only detected from C2, then the organism is homozygous for the G allele.

Background signals due to hybridization of transcription cassettes (containing P1 and P2 sequences) to C1 and C2 tag sequences on the chip can be minimized by attaching the transcription cassettes to a solid surface, e.g. using biotin-labelled transcription cassettes and binding the cassette to a SA-coated plate or SA-coated beads. The transcription products generated from the transcription cassettes that have ligated 5′ flaps (product of the first or second ICR) are free in solution in the SA-coated plate and can be transferred to the universal detector. Alternately, the 5′-end of the ICR probe or ICR hairpin cassette (serial ICR) could be 5′ exonuclease-protected (e.g., with phosphorothioate nucleotides) as well as the 3′-end of the transcription hairpin cassette. Uncleaved ICR probes and ICR hairpin cassettes could be digested with a 3′-exonuclease prior to generation of the transcription products. Another embodiment of this format could use a development reagent using methods described previously by inclusion of an additional portion of sequence upstream of the identifier tag sequence that is present in the transcription hairpin cassette.

### Example 7

### SNP Genotyping Using ICR Followed by Polymerization and Transcription

In order to determine the genotype of a diploid organism, a genomic DNA sample is assayed to determine which polymorphic nucleotides are present for a particular SNP site. As illustrated in FIG. 9, a target SNP may have T or G at the polymorphic position. In this assay, ICR or serial ICR is carried out using genomic DNA or PCR product (FIG. 9, Step 1) using any of the methods as previously described. The 5′ flap released from the ICR or second ICR of a serial ICR is hybridized to a single stranded template that contains, in the 3′ to 5′ direction, a region complementary to the 5′ flap, a T7 promoter region, and a region with sequence complementary to the identifier tag for that allele. When the 5′ flap binds to the template, it serves as aprimer in a polymerization reaction that generates double-stranded copies of the template (Step 2, indicated by wavy line). Klenow fragment, at 37° C., is one such polymerase suitable for this polymerization step. The polymerization step generates a double stranded T7 promoter site, which T7 polymerase can then use to generate RNA molecules containing identifier tags (FIG. 9, Step 3, indicated as P1 RNA tags and P2 RNA tags). These RNA molecules, containing identifier tags, are tagged RNA molecules suitable for use in a universal tag assay. Another embodiment of this format could use a development reagent using methods described previously and inclusion of an additional portion of sequence downstream of the tag sequence that is present in the 5′-flap/T7/Tag template. Alternate versions that can be contemplated by one skilled in the art are use of other RNA polymerases and their respective promoter sequences (e.g., SP6 and T3).

Hybridization of tagged RNA molecules to complementary detection probes is detected by electrochemical readout (Step 4), e.g., by incubation with a universal detector in the presence of a ruthenium complex. As shown in FIG. 9, a hybridization signal from detection probe C1 reports that the P1 identifier tag was generated and bound to C1, which indicates that the T allele is present in the sample. A hybridization signal from detection probe C2 reports that the P2 identifier tag was generated and bound to C2, which indicates that the G allele is present in the sample. Hybridization signals from both C1 and C2 indicate that both the T allele and the G allele are present in the sample, i.e., the organism from which the genomic DNA was obtained is heterozygous at this SNP. When a signal is only detected from C1, then the organism is homozygous for the T allele, and when a signal is only detected from C2, then the organism is homozygous for the G allele.

1. A method of detecting a target nucleotide sequence in a sample, comprising:
a) providing
i) template comprising at least one target nucleotide sequence;
ii) at least one probe oligonucleotide comprising a 3′ portion complementary to a portion of said template comprising target nucleotide sequence and a 5′ portion not complementary to said template comprising target nucleotide sequence;
iii) at least one upstream oligonucleotide complementary to a portion of said template comprising target nucleotide sequence wherein said portion is 3′ to and partially overlapping the 3′ portion of said template comprising target nucleotide sequence complementary to said probe oligonucleotide; and
iv) a cleaving agent;
b) mixing, in any order, said template comprising target nucleotide sequence, said probe oligonucleotide, said upstream oligonucleotide, and said cleaving agent under reaction conditions such that said 3′ portion of said probe oligonucleotide is annealed to said template and said upstream oligonucleotide is annealed to said template so as to create a cleavage structure wherein said probe oligonucleotide and said upstream oligonucleotide overlap by at least one nucleotide;
c) cleaving said cleavage structure to release a 5′ flap, comprising cleaving said probe oligonucleotide at a position one nucleotide 3′ of the portion of said probe oligonucleotide that overlaps said upstream oligonucleotide, releasing said 5′ flap comprising said 5′ portion of said probe oligonucleotide not complementary to contiguous target nucleotide sequence and further comprising any overlapping nucleotide;
d) utilizing said 5′ flap as a reagent in at least one subsequent reaction to generate at least one tagged molecule comprising an identifier tag chosen to serve as an identifier for said target nucleotide sequence;
e) contacting said at least one tagged molecule with a universal detector comprising at least one complementary detection probe coupled to a detection means, wherein said complementary detection probe comprises sequence complementary to said identifier tag; and
f) measuring hybridization of said identifier tag to said complementary detection probe, wherein said hybridization indicates the presence of the corresponding target nucleotide sequence in said sample.

2. The method of claim 1, wherein said template is DNA or RNA.

3. The method of claim 2, wherein said template comprises any one of genomic DNA, polymerase chain reaction (PCR) product, cDNA, rolling circle (RC) amplification product, mRNA, or viral RNA.

4. The method of claim 1, wherein said reaction conditions comprise a reaction temperature between approximately 40 and approximately 75 degrees Centigrade.

5. The method of claim 1, wherein multiple probe oligonucleotides are cleaved and multiple 5′ flaps are released.

6. The method of claim 1, wherein said method is used to detect variant sequences of said target nucleotide sequence.

7. The method of claim 1, wherein said probe oligonucleotide and said upstream oligonucleotide overlap by one nucleotide.

8. The method of claim 7, wherein said method is used to determine one or more polymorphic nucleotides in a single nucleotide polymorphism (SNP), said method comprising providing at least one probe oligonucleotide and at least one upstream oligonucleotide complementary to and overlapping at the polymorphic nucleotide of a first allele of said SNP, and further providing at least one probe oligonucleotide and at least one upstream oligonucleotide complementary to and overlapping at the polymorphic nucleotide of a second allele of said SNP, wherein said oligonucleotide probes and upstream oligonucleotides anneal to said template so as to create a distinct cleavage structure for each allele of said SNP, with the result that an allele-specific 5′ flap is released from each cleavage structure if the corresponding allele of said SNP is present in said sample and with the further result that each said allele-specific 5′ flap released from said cleavage structure generates an allele-specific tagged molecule, wherein each said allele-specific tagged molecule comprises the identifier tag chosen to serve as the identifier for the corresponding allele.

9. The method of claim 8, wherein said SNP comprises more than two alleles, further comprising providing oligonucleotide probe and upstream oligonucleotides complementary to and overlapping at each said allele of said SNP.

10. The method of claim 1, wherein a plurality of target nucleotide sequences in a sample are detected, said method comprising:
a) providing template comprising a plurality of target nucleotide sequences, and further providing at least one probe oligonucleotide and at least one upstream oligonucleotide complementary to a portion of template comprising each target nucleotide sequence of said plurality of target nucleotide sequences;
b) mixing said oligonucleotide probes and upstream oligonucleotides with said template under reaction conditions such that each oligonucleotide probe and upstream oligonucleotide will anneal to said template to create a distinct cleavage structure for each target nucleotide sequence, with the result that at least one distinct 5′ flap corresponding to each said target nucleotide sequence of said plurality of target nucleotide sequences is released from each distinct cleavage structure if the corresponding target nucleotide sequence is present in said sample;
c) utilizing each said distinct 5′ flap as a reagent in at least one subsequence reaction to generate at least one distinct tagged molecule corresponding to each said target nucleotide sequence of said plurality of target nucleotide sequences, wherein each said distinct tagged molecule comprises the identifier tag chosen to serve as an identifier to each corresponding target nucleotide sequence; and
d) measuring hybridization of each said identifier tag corresponding to each said target nucleotide sequence of said plurality of target nucleotide sequences, wherein each said hybridization indicates the presence of said corresponding target nucleotide sequence in said sample.

11. The method of claim 1, wherein said cleaving agent is a 5′ endonuclease.

12. A method of detecting a target nucleotide sequence in a sample, comprising:
a) performing a first invasion cleavage reaction (ICR) as in steps a) to c) of claim 1, releasing a first 5′ flap;
b) performing a second invasion cleavage reaction (ICR) comprising:
i) providing a second template, a second probe oligonucleotide, and said first 5′ flap, wherein said second probe oligonucleotide comprises a 3′ portion complementary to a portion of said second template and a 5′ portion not complementary to said second template, and further wherein said first 5′ flap is 5′ to and partially overlapping the 3′ portion of said second template comprising target nucleotide sequence complementary to said second probe oligonucleotide;
ii) mixing, in any order, said second template, said second probe oligonucleotide, and said first 5′ flap, under reaction conditions such that said 3′ portion of said second probe oligonucleotide is annealed to said second template and said first 5′ flap is annealed to said second template so as to create a cleavage structure wherein said second probe oligonucleotide and said first 5′ flap overlap by at least one nucleotide;
iii) cleaving said cleavage structure to release a second 5′ flap, comprising cleaving said second probe oligonucleotide at a position one nucleotide 3′ of the portion of said second probe oligonucleotide that overlaps said first 5′ flap, releasing said second 5′ flap comprising said 5′ portion of said second probe oligonucleotide not complementary to contiguous target nucleotide sequence and further comprising any overlapping nucleotide;
c) utilizing said second 5′ flap as a reagent in at least one subsequent reaction to generate at least one tagged molecule comprising an identifier tag chosen to serve as an identifier for said target nucleotide sequence;
d) contacting said at least one tagged molecule with a universal detector comprising at least one complementary detection probe coupled to a detection means, wherein said complementary detection probe comprises sequence complementary to said identifier tag; and
e) measuring hybridization of said identifier tag to said complementary detection probe, wherein said hybridization indicates the presence of the corresponding target nucleotide sequence in said sample.

13. The method of claim 12, wherein no additional cleaving agent is provided for said second ICR.

14. The method of claim 12, wherein an additional cleaving agent is provided for said second ICR.

15. The method of claim 12, wherein said second template and said second probe oligonucleotide are provided as a hairpin cassette.

16. The method of claim 15, wherein said hairpin cassette is addressably labelled, such that said labelled hairpin cassette can be manipulated through a label-binding moiety.

17. The method of claim 16, wherein said hairpin cassette is biotinylated.

18. The method of claim 17, wherein said biotinylated hairpin cassette is contacted with streptavidin coupled to a solid support, such that said biotinylated hairpin cassette can be removed from the reaction mixture.

19. The method of claim 12, wherein said tagged molecule comprises an identifier tag chosen to serve as an identifier for said target nucleotide sequence, and a development reagent.

20. The method of claim 19, wherein said contacting said tagged molecule with said universal detector is carried out in the presence of a development reagent binding moiety to generate a functional development reagent, further wherein said measuring hybridization of said identifier tag to said complementary detection probe includes measuring the contribution of said functional development reagent to the signal being measured.

21. The method of claim 20, wherein said development reagent binding moiety comprises an oligonucleotide complementary to said development reagent.

22. The method of claim 21, wherein said development reagent binding moiety is attached to at least one said detection probe.

23. The method of claim 12, wherein said utilizing said second 5′ flap as a reagent in at least one subsequent reaction to generate at least one tagged molecule comprises using said second 5′ flap is used as a polymerization primer for rolling circle (RC) amplification of at least one circular oligonucleotide, said circular oligonucleotide comprising sequence complementary to said second 5′ flap and sequence complementary to an identifier tag chosen to serve as an identifier for said target nucleotide sequence, such that said RC amplification generates at least one tagged molecule comprising multiple copies of said identifier tag.

24. The method of claim 23, wherein said method is used to determine one or more polymorphic nucleotides in a single nucleotide polymorphism (SNP).

25. The method of claim 23, wherein said method is used to detect a plurality of target nucleotide sequences in said sample.

26. The method of claim 23, wherein said tagged molecule is trimmed to generate shorter tagged molecules comprising one copy of said identifier tag.

27. The method of claim 12, further comprising transcribing said second 5′ flap to generate RNA tagged molecules, wherein said method comprises:
a) providing at least one distinct second 5′ flap comprising sequence complementary to the identifier tag chosen to serve as the identifier for each said target nucleotide sequence in said sample;
b) contacting each said distinct second 5′ flap with a template comprising a double stranded RNA polymerase promoter and a single stranded portion of sequence complementary to said second 5′ flap;
c) allowing each said second 5′ flap to anneal to said sequence complementary to said second 5′ flap;
d) ligating each said annealed second 5′ flap to contiguous sequence;
e) transcribing each said annealed second 5′ flap in the presence of RNA polymerase, generating at least one RNA tagged molecule comprising the identifier tag for each said target nucleotide sequence in said sample; and
f) contacting said at least one tagged RNA molecule with a universal detector comprising at least one complementary detection probe coupled to a detection means, wherein each said complementary detection probe comprises sequence complementary to each said identifier tag, and measuring hybridization of each said identifier tag to each said complementary detection probe, wherein said hybridization indicates the presence of the corresponding target nucleotide sequence in the sample.

28. The method of claim 27, wherein said template comprising a double stranded RNA polymerase promoter and a single stranded portion of sequence complementary to said second 5′ flap is a hairpin cassette.

29. The method of claim 28, wherein said hairpin cassette is addressably labelled, such that said labelled hairpin cassette can be manipulated through a label-binding moiety.

30. The method of claim 29, wherein said hairpin cassette is biotinylated.

31. The method of claim 30, wherein said biotinylated hairpin cassette is contacted with streptavidin coupled to a solid support, such that said biotinylated hairpin cassette can be removed from the reaction mixture.

32. The method of claim 27, wherein said method is used to determine one or more polymorphic nucleotides in a single nucleotide polymorphism (SNP).

33. The method of claim 27, wherein said method is used to detect a plurality of target nucleotide sequences in said sample.

34. The method of claim 12, further comprising transcribing said second 5′ flap to generate RNA tagged molecules, wherein said method comprises:
a) providing at least one distinct second 5′ flap corresponding to each said target nucleotide sequence in said sample;
b) contacting each said distinct second 5′ flap with at least one single stranded template, wherein each template comprises a portion of sequence complementary to one said distinct second 5′ flap, a portion of sequence encoding one strand of RNA polymerase promoter, and at least one copy of the identifier tag chosen to serve as the identifier for said target nucleotide sequence corresponding to said distinct second 5′ flap;
c) allowing each said second 5′ flap to anneal to said template sequence complementary to said second 5′ flap, forming a double-stranded DNA polymerase binding site;
d) generating a double stranded copy of said template by DNA polymerase binding to said double stranded site and extension of said annealed second 5′ flap;
e) contacting said double stranded copy of said template with RNA polymerase, allowing RNA polymerase to transcribe sequence downstream of said RNA polymerase promoter, thereby generating RNA tagged molecules comprising the identifier tag for each said target nucleotide sequence in said sample; and
f) contacting said at least one tagged RNA molecule with a universal detector comprising at least one complementary detection probe coupled to a detection means, wherein each said complementary detection probe comprises sequence complementary to each said identifier tag, and measuring hybridization of each said identifier tag to each said complementary detection probe, wherein said hybridization indicates the presence of the corresponding target nucleotide sequence in the sample.

35. The method of claim 34, wherein said method is used to determine one or more polymorphic nucleotides in a single nucleotide polymorphism (SNP).

36. The method of claim 34, wherein said method is used to detect a plurality of target nucleotide sequences in said sample.

37-91. (canceled)

92. A method of detecting a target nucleotide sequence in a sample comprising:
obtaining a sample comprising template, said template comprising at least one target nucleotide sequence;
obtaining an oligonucleotide probe comprising a 3′ portion complementary to a portion of said template comprising target nucleotide sequence and a 5′ portion not substantially complementary to a portion of said template comprising target nucleotide sequence;
obtaining an upstream oligonucleotide complementary to a portion of said template comprising target nucleotide sequence, wherein said portion is 3′ to and partially overlapping the 3′ portion of said template comprising target nucleotide sequence complementary to said probe nucleic acid;
contacting said sample with said oligonucleotide probe and said upstream oligonucleotide under conditions which permit hybridization of said oligonucleotide probe and said upstream oligonucleotide to said template comprising target nucleotide sequence, whereby the 5′ portion of said oligonucleotide probe forms a 5′ flap that is not substantially complementary to a portion of said template comprising target nucleotide sequence;
cleaving said 5′ flap from said oligonucleotide probe; and
detecting said cleaved 5′ flap.

93. The method of claim 92, wherein multiple probe oligonucleotides are cleaved and multiple 5′ flaps are released.

94. The method of claim 92, wherein an endonuclease mediates said cleaving step.

95. The method of claim 94, wherein said endonuclease comprises a 5′ endonuclease.

96. The method of claim 92, wherein the step of detecting said cleaved 5′ flap comprises using said cleaved 5′ flap as a primer in a rolling circle amplification procedure.

97. The method of claim 92, wherein said cleaved 5′ flap comprises a promoter or a sequence complementary to a promoter and the step of detecting said cleaved 5′ flap comprises initiating transcription from said promoter.
